SEQUENCE LISTING (75 sequences)

SEQ ID NO: 1 - DNA, 20 bp, Artificial Sequence
  Description of Artificial Sequence Synthetic oligonucleotide
ttacttctgacaacgatcgg

SEQ ID NO: 2 - DNA, 20 bp, Artificial Sequence
  Description of Artificial Sequence Synthetic oligonucleotide
atcgaactggatctcaacag

SEQ ID NO: 3 - DNA, 20 bp, Artificial Sequence
  Description of Artificial Sequence Synthetic oligonucleotide
tggaagatcaggatatgtgg

SEQ ID NO: 4 - DNA, 60 bp, Artificial Sequence
  Description of Artificial Sequence Synthetic primer
aaactcccacatggattcgaaatcaatctaaagtatatatgagtaaacttggtctgacag

SEQ ID NO: 5 - DNA, 60 bp, Artificial Sequence
  Description of Artificial Sequence Synthetic primer
cccaagtaccgccacctaaaaaacttcatttttaatttaaaaggatctaggtgaagatcc

SEQ ID NO: 6 - DNA, 39 bp, Artificial Sequence
  Description of Artificial Sequence Synthetic primer
taaattaaaaatgaagttttttaggtggcggtacttggg

SEQ ID NO: 7 - DNA, 48 bp, Artificial Sequence
  Description of Artificial Sequence Synthetic primer
tatatactttagattgatttcgaatccatgtgggagtttattcttgac

SEQ ID NO: 8 - DNA, 43 bp, Artificial Sequence
  Description of Artificial Sequence Synthetic primer
ttacttctgacaacgatcgggttttagagctagaaatagcaag

SEQ ID NO: 9 - DNA, 23 bp, Artificial Sequence
  Description of Artificial Sequence Synthetic primer
tttataacctccttagagctcga

SEQ ID NO: 10 - DNA, 21 bp, Artificial Sequence
  Description of Artificial Sequence Synthetic primer
ccaattgtccatattgcatca

SEQ ID NO: 11 - DNA, 43 bp, Artificial Sequence
  Description of Artificial Sequence Synthetic primer
ccgatcgttgtcagaagtaagtgctcagtatctctatcactga

SEQ ID NO: 12 - DNA, 43 bp, Artificial Sequence
  Description of Artificial Sequence Synthetic primer
atcgaactggatctcaacaggttttagagctagaaatagcaag

SEQ ID NO: 13 - DNA, 43 bp, Artificial Sequence
  Description of Artificial Sequence Synthetic primer
ctgttgagatccagttcgatgtgctcagtatctctatcactga

SEQ ID NO: 14 - DNA, 48 bp, Artificial Sequence
  Description of Artificial Sequence Synthetic primer
tacatcgaactggatctcaacttccctatcagtgatagagattgacat

SEQ ID NO: 15 - DNA, 48 bp, Artificial Sequence
  Description of Artificial Sequence Synthetic primer
agccgtagttaggccaccacaattccagaaatcatccttagcgaaagc

SEQ ID NO: 16 - DNA, 39 bp, Artificial Sequence
  Description of Artificial Sequence Synthetic primer
taaggatgatttctggaattgtggtggcctaactacggc

SEQ ID NO: 17 - DNA, 47 bp, Artificial Sequence
  Description of Artificial Sequence Synthetic primer
ctctatcactgatagggaagttgagatccagttcgatgtaacccact

SEQ ID NO: 18 - DNA, 39 bp, Artificial Sequence
  Description of Artificial Sequence Synthetic primer
ggcggataaagttgcaggacttgggcccgaacaaaaact

SEQ ID NO: 19 - DNA, 43 bp, Artificial Sequence
  Description of Artificial Sequence Synthetic primer
tctcaaggatcttaccgctggcttcaaaaaaagcaccgactcg

SEQ ID NO: 20 - DNA, 44 bp, Artificial Sequence
  Description of Artificial Sequence Synthetic primer
gtcggtgctttttttgaagccagcggtaagatccttgagagttt

SEQ ID NO: 21 - DNA, 41 bp, Artificial Sequence
  Description of Artificial Sequence Synthetic primer
gagtttttgttcgggcccaagtcctgcaactttatccgcct

SEQ ID NO: 22 - DNA, 47 bp, Artificial Sequence
  Description of Artificial Sequence Synthetic primer
tggacgagctgtacaagtaacttccctatcagtgatagagattgaca

SEQ ID NO: 23 - DNA, 46 bp, Artificial Sequence
  Description of Artificial Sequence Synthetic primer
cctcgcccttgctcaccatttgagatccagttcgatgtaacccact

SEQ ID NO: 24 - DNA, 38 bp, Artificial Sequence
  Description of Artificial Sequence Synthetic primer
tacatcgaactggatctcaaatggtgagcaagggcgag

SEQ ID NO: 25 - DNA, 45 bp, Artificial Sequence
  Description of Artificial Sequence Synthetic primer
ctctatcactgatagggaagttacttgtacagctcgtccatgccg

SEQ ID NO: 26 - DNA, 43 bp, Artificial Sequence
  Description of Artificial Sequence Synthetic primer
tggaagatcaggatatgtgggttttagagctagaaatagcaag

SEQ ID NO: 27 - DNA, 43 bp, Artificial Sequence
  Description of Artificial Sequence Synthetic primer
ccacatatcctgatcttccagtgctcagtatctctatcactga

SEQ ID NO: 28 - DNA, 50 bp, Artificial Sequence
  Description of Artificial Sequence Synthetic primer
atctggaagatcaggatatgcttccctatcagtgatagagattgacatcc

SEQ ID NO: 29 - DNA, 48 bp, Artificial Sequence
  Description of Artificial Sequence Synthetic primer
ggcctcttcgctattacgccaattccagaaatcatccttagcgaaagc

SEQ ID NO: 30 - DNA, 40 bp, Artificial Sequence
  Description of Artificial Sequence Synthetic primer
taaggatgatttctggaattggcgtaatagcgaagaggcc

SEQ ID NO: 31 - DNA, 48 bp, Artificial Sequence
  Description of Artificial Sequence Synthetic primer
ctctatcactgatagggaagcatatcctgatcttccagataactgccg

SEQ ID NO: 32 - DNA, 47 bp, Artificial Sequence
  Description of Artificial Sequence Synthetic primer
atggtcaggtcatggatgagttgggcccgaacaaaaactcatctcag

SEQ ID NO: 33 - DNA, 41 bp, Artificial Sequence
  Description of Artificial Sequence Synthetic primer
aaaatgccgctcatccgccagcttcaaaaaaagcaccgact

SEQ ID NO: 34 - DNA, 36 bp, Artificial Sequence
  Description of Artificial Sequence Synthetic primer
gtcggtgctttttttgaagctggcggatgagcggca

SEQ ID NO: 35 - DNA, 42 bp, Artificial Sequence
  Description of Artificial Sequence Synthetic primer
gagtttttgttcgggcccaactcatccatgacctgaccatgc

SEQ ID NO: 36 - DNA, 50 bp, Artificial Sequence
  Description of Artificial Sequence Synthetic primer
tggacgagctgtacaagtaacttccctatcagtgatagagattgacatcc

SEQ ID NO: 37 - DNA, 48 bp, Artificial Sequence
  Description of Artificial Sequence Synthetic primer
tcctcgcccttgctcaccatcatatcctgatcttccagataactgccg

SEQ ID NO: 38 - DNA, 38 bp, Artificial Sequence
  Description of Artificial Sequence Synthetic primer
atctggaagatcaggatatgatggtgagcaagggcgag

SEQ ID NO: 39 - DNA, 44 bp, Artificial Sequence
  Description of Artificial Sequence Synthetic primer
accagtagaaacagacgaagaatcgtggtggcctaactacggct

SEQ ID NO: 40 - DNA, 37 bp, Artificial Sequence
  Description of Artificial Sequence Synthetic primer
ttgctcaccatttgagatccagttcgatgtaacccac

SEQ ID NO: 41 - DNA, 37 bp, Artificial Sequence
  Description of Artificial Sequence Synthetic primer
ctgtacaagtaacagcggtaagatccttgagagtttt

SEQ ID NO: 42 - DNA, 46 bp, Artificial Sequence
  Description of Artificial Sequence Synthetic primer
tatcaaagggaaaactgtccataccgtcctgcaactttatccgcct

SEQ ID NO: 43 - DNA, 27 bp, Artificial Sequence
  Description of Artificial Sequence Synthetic primer
ggatctcaaatggtgagcaagggcgag

SEQ ID NO: 44 - DNA, 37 bp, Artificial Sequence
  Description of Artificial Sequence Synthetic primer
atcttaccgctgttacttgtacagctcgtccatgccg

SEQ ID NO: 45 - DNA, 32 bp, Artificial Sequence
  Description of Artificial Sequence Synthetic primer
agacgcgaattatttttgatggcgttaactcg

SEQ ID NO: 46 - DNA, 24 bp, Artificial Sequence
  Description of Artificial Sequence Synthetic primer
gaagggctggtcttcatccacgcg

SEQ ID NO: 47 - DNA, 22 bp, Artificial Sequence
  Description of Artificial Sequence Synthetic primer
gccgctggcgacctgcgtttca

SEQ ID NO: 48 - DNA, 24 bp, Artificial Sequence
  Description of Artificial Sequence Synthetic primer
gagtattcaacatttccgtgtcgc

SEQ ID NO: 49 - DNA, 50 bp, Artificial Sequence
  Description of Artificial Sequence Synthetic primer
cagggtagccagcagcatcccttccctatcagtgatagagattgacatcc

SEQ ID NO: 50 - DNA, 43 bp, Artificial Sequence
  Description of Artificial Sequence Synthetic primer
tgttccggatctgcatcgcagcttcaaaaaaagcaccgactcg

SEQ ID NO: 51 - DNA, 39 bp, Artificial Sequence
  Description of Artificial Sequence Synthetic primer
gtcggtgctttttttgaagctgcgatgcagatccggaac

SEQ ID NO: 52 - DNA, 38 bp, Artificial Sequence
  Description of Artificial Sequence Synthetic primer
ctctatcactgatagggaagggatgctgctggctaccc

SEQ ID NO: 53 - DNA, 40 bp, Artificial Sequence
  Description of Artificial Sequence Synthetic primer
cagggtagccagcagcatccgtggtggcctaactacggct

SEQ ID NO: 54 - DNA, 41 bp, Artificial Sequence
  Description of Artificial Sequence Synthetic primer
tgttccggatctgcatcgcagtcctgcaactttatccgcct

SEQ ID NO: 55 - DNA, 37 bp, Artificial Sequence
  Description of Artificial Sequence Synthetic primer
ggcggataaagttgcaggactgcgatgcagatccgga

SEQ ID NO: 56 - DNA, 39 bp, Artificial Sequence
  Description of Artificial Sequence Synthetic primer
agccgtagttaggccaccacggatgctgctggctaccct

SEQ ID NO: 57 - DNA, 44 bp, Artificial Sequence
  Description of Artificial Sequence Synthetic primer
ctctatcactgatagggaagttgagatccagttcgatgtaaccc

SEQ ID NO: 58 - DNA, 36 bp, Artificial Sequence
  Description of Artificial Sequence Synthetic primer
taaggatgatttctggaatttgaggacgaggtggcc

SEQ ID NO: 59 - DNA, 47 bp, Artificial Sequence
  Description of Artificial Sequence Synthetic primer
tgacggccacctcgtcctcaaattccagaaatcatccttagcgaaag

SEQ ID NO: 60 - DNA, 45 bp, Artificial Sequence
  Description of Artificial Sequence Synthetic primer
gtcggtgctttttttgaagccagcggtaagatccttgagagtttt

SEQ ID NO: 61 - DNA, 53 bp, Artificial Sequence
  Description of Artificial Sequence Synthetic primer
tcaggtttgtgccaataccagtagaaacagacgaagaatcggcgtaatagcga

SEQ ID NO: 62 - DNA, 44 bp, Artificial Sequence
  Description of Artificial Sequence Synthetic primer
aaaatgccgctcatccgccattacttgtacagctcgtccatgcc

SEQ ID NO: 63 - DNA, 36 bp, Artificial Sequence
  Description of Artificial Sequence Synthetic primer
tggacgagctgtacaagtaatggcggatgagcggca

SEQ ID NO: 64 - DNA, 56 bp, Artificial Sequence
  Description of Artificial Sequence Synthetic primer
gttcaccgttacatatcaaagggaaaactgtccataccctcatccatgacctgacc

SEQ ID NO: 65 - DNA, 25 bp, Artificial Sequence
  Description of Artificial Sequence Synthetic primer
atcccgtgacaggtcattcagactg

SEQ ID NO: 66 - DNA, 18 bp, Artificial Sequence
  Description of Artificial Sequence Synthetic primer
gatttaatctgtatcagg

SEQ ID NO: 67 - DNA, 23 bp, Artificial Sequence
  Description of Artificial Sequence Synthetic oligonucleotide
tagcttgacctagagttgtcgcc

SEQ ID NO: 68 - DNA, 40 bp, Artificial Sequence
  Description of Artificial Sequence Synthetic oligonucleotide
tccctatcagtgatagagatactgagcacatcgaactgga

SEQ ID NO: 69 - DNA, 40 bp, Artificial Sequence
  Description of Artificial Sequence Synthetic oligonucleotide
acatcgaactggatctcaacaggttttagagctagaaata

SEQ ID NO: 70 - DNA, 40 bp, Artificial Sequence
  Description of Artificial Sequence Synthetic oligonucleotide
acatcgaactggatctcaacagcggtaagatccttgagag

SEQ ID NO: 71 - DNA, 20 bp, Artificial Sequence
  Description of Artificial Sequence Synthetic oligonucleotide
atctggaagatcaggatatg

SEQ ID NO: 72 - DNA, 20 bp, Artificial Sequence
  Description of Artificial Sequence Synthetic oligonucleotide
acatccctatcagtgataga

SEQ ID NO: 73 - DNA, 31 bp, Unknown
  Description of Unknown genomic sequence
tgtgggttttggacactggaaccgtgggcat

SEQ ID NO: 74 - RNA, 20 nt, Artificial Sequence
  Description of Artificial Sequence Synthetic oligonucleotide
gguuuuggacacuggaaccg

SEQ ID NO: 75 - DNA, 33 bp, Unknown
  Description of Unknown genomic sequence
ggatgcccacggttccagtgtccaaaacccaca

(19) United States
(12) Patent Application Publication
(10) Pub. No.: US 2023/0046246 A1
(43) Pub. Date: Feb. 16, 2023

(54) Reducing Antibiotic Resistance in Bacteria Using Pro-Active Genetics

(71) Applicant: The Regents of the University of California, Oakland, CA (US)

(72) Inventors: Ethan Bier, San Diego, CA (US); Victor Nizet, La Jolla, CA (US); Jonathan Andres Valderrama, La Jolla, CA (US); Surashree Kulkarni, La Jolla, CA (US)

(21) Appl. No.: 17/783,255

(22) PCT Filed: Dec. 14, 2020
(86) PCT No.: PCT/US2020/064871

§ 371 Date: Jun. 7, 2022

Related U.S. Application Data
(60) Provisional application No. 62/947,827, filed on Dec. 13, 2019.

(51) Int. Cl. C12N 15/10; C12N 15/70; C12N 15/82

(52) U.S. Cl. CPC C12N 15/102; C12N 15/70; C12N 15/8209; C12N 2310/20

(57) ABSTRACT

CRISPR-based gene-drive system for inhibiting antibiotic resistance of bacteria, including Escherichia coli that efficiently copies a gRNA cassette and adjacent cargo that are flanked with sequences homologous to the targeted gRNA/Cas9 cleavage site. This “pro-active” genetic system (Pro-AG) functionally inactivates an antibiotic resistance marker on a high copy number plasmid with greater efficiency than control CRISPR-based methods. Pro-AG can effectively edit large plasmids or single-copy genomic targets, or introduce functional genes, with numerous applications to biotechnology and biomedicine.

### CROSS REFERENCE TO RELATED APPLICATIONS

This application claims the priority benefit of U.S. Provisional Application No. 62/947,827, filed Dec. 13, 2019, which application is incorporated herein by reference.

### GOVERNMENT SPONSORSHIP

This invention was made with government support under grant No. GM117321 awarded by the National Institutes of Health. The government has certain rights in the invention.

### TECHNICAL FIELD

The present invention relates to reducing antibiotic resistance in bacteria using CRISPR systems.

### BACKGROUND

Bacterial resistance to antibiotics is one of the greatest current threats to human health. Some experts predict that by mid-century 10 million people a year will die of antibiotic resistant infections, more than that caused by all vector-borne diseases combined. This crisis has arisen in part because of over prescription of antibiotics and because of their ubiquitous use as in animal food production such as fish farms and growth stimulants for livestock. A troubling result of these practices is the increasing prevalence of antibiotic resistant strains of bacteria in the environment (e.g., fish ponds, feedlots, sewers).

One reason that antibiotic resistance has become so pervasive is that it can be transferred between bacteria (even between different species). One common form of DNA elements carrying antibiotic resistance genes are circular forms of DNA called plasmids, which can exist in many copies per cell (e.g., high-copy number plasmids have more than 50 copies per bacterial cell). Because of their high copy number, such plasmids produce great amounts of the gene products conferring antibiotic resistance and hence present a formidable challenge to antibiotic treatment therapies.

Synthetic gene-drive elements based on bacterial-derived CRISPR components have been developed in diploid organisms including yeast¹, insects²⁻⁴, and mammals⁵. The salient feature of these “active genetic” systems is that a guide RNA (gRNA) is directly flanked by sequences homologous to the genomic site it targets for Cas9-mediated cleavage. When double-stranded DNA breaks are induced in the germline by the gRNA/Cas9 complex, the gRNA together with any linked cargo sequences are then copied into the break via the homology directed repair (HDR) pathway⁶. Such gene conversion events can greatly bias transmission of the gRNA cassette so that it is inherited by nearly all progeny.

Echoing their functional CRISPR origins, synthetic bipartite gRNA/Cas9 systems have been developed in bacteria⁷⁻⁹, including targeting of plasmid-encoded antibiotic resistance determinants¹⁰. Indeed, since plasmid-borne antibiotic resistance genes and virulence determinants are important in the pathogenesis of many human bacterial infections, inactivation or replacement of such multi-copy targets could greatly impact the success or failure of treatment interventions^{11,12}.

### SUMMARY OF THE INVENTION

The disclosure of the present invention provides constructs, systems and methods of inhibiting antibiotic resistance in bacteria comprising modifying a bacterial genome with a prokaryotic-active genetics (Pro-AG) system. The methods are applicable to direct application to bacteria, as well as indirectly to subjects and environments suspected of containing such bacteria.

In embodiments, this “active genetics” has been applied in bacteria to propagate a guide RNA (gRNA) cassette targeting, for example, the beta-lactamase gene (bla). A donor plasmid initially provides the gRNA cassette flanked with bla sequences adjacent to the cut site (homology arms). Following induction of Cas9 and a DNA repair system (λRed), the gRNA element copies onto the AR bla high-copy number plasmid with extraordinarily high efficiency and precision. This precise “editing” does not destroy the target plasmid and results in ˜100,000-fold reduction in the incidence of AR in the bacterial population, representing >100-fold improvement over the standard CRISPR approach of simply cutting the targeted plasmid. Pro-AG is driven by a self-amplifying positive feed-back loop sustained largely by increasing levels of the gRNA bearing cassette, a limiting element in this system. The systems, compositions, and methods of this invention provide potent new tools to help prevent or treat chronic AR bacterial infections, and potentially to remove AR from environmental sites of concern.

In embodiments, the prokaryotic-active genetics system comprises a method of reducing or inhibiting antibiotic resistance in bacteria comprising modifying a bacterial plasmid gene for antibiotic resistance with a prokaryotic-active genetics (Pro-AG) system. In embodiments, the Pro-AG system comprises a first plasmid encoding an inducible Cas9 protein. In embodiments, the Pro-AG system further comprises a second plasmid encoding: (i) a guide ribonucleic acid (gRNA) cassette comprising a promoter for constitutive expression of a gRNA having a sequence that hybridizes to a target genomic sequence on a target plasmid in the bacteria, wherein the target genomic sequence in the bacteria confers antibiotic resistance; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iii) an inducible λRed DNA repair cassette. The invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby modifying the bacterial plasmid gene to inhibit antibiotic resistance.

In embodiments, the invention provides that copies of the guide RNA cassette increase via a positive feedback loop allowing for self-amplification of the Pro-AG system. In embodiments, the invention provides that the first plasmid is present in a lower copy number than the second plasmid and the bacterial plasmid. In embodiments, the invention provides that the bacterial gene for antibiotic resistance is a beta-lactamase gene. In embodiments, the invention provides that the antibiotic is ampicillin or gentamicin. In embodiments, the invention provides that the bacteria is Escherichia coli.

In embodiments, the invention provides that the Cas9 protein is induced with anhydrotetracycline. In embodiments, the invention provides that the λRed DNA repair cassette is induced with arabinose. In embodiments, the invention provides that a Tet promoter drives constitutive expression of the gRNA.

In embodiments, the invention provides that the bacteria is in a subject. In embodiments, the invention provides that the bacteria is on a solid surface or in a liquid.

In embodiments, the invention provides that the second plasmid further comprises at least one cargo sequence, which is inserted into the bacterial plasmid. In embodiments, the invention provides that the at least one cargo sequence encodes GFP. In embodiments, the invention provides that the at least one cargo sequence is not flanked by the first and second homology sequences on the second plasmid.

In embodiments, the invention provides that the second plasmid comprises a dual Pro-AG system further comprising, (i) a further guide ribonucleic acid (gRNA) cassette comprising a further promoter for constitutive expression of a further gRNA having a further sequence that hybridizes to a further target genomic sequence on the target plasmid in the bacteria; and (ii) a further first homology arm and a further second homology arm each flanking opposite ends of the further gRNA cassette in the second plasmid, wherein the further first homology arm and the further second homology arm have sequences that hybridize to respective sequences on opposite sides of a cut site for the Cas9 protein on the further target genomic sequence.

In embodiments, the invention provides that the second plasmid comprises a nested Pro-AG system further comprising, (i) a further gRNA having a further sequence that hybridizes to a further target genomic sequence on the target plasmid in the bacteria, wherein the further guide RNA is adjacent to the first homology arm outside the gRNA cassette; and (ii) a further first homology arm outside the gRNA cassette on a side of the plasmid adjacent the second homology arm, and a further second homology arm outside the gRNA cassette on an opposite side of the plasmid adjacent the further gRNA, wherein the further first homology arm and the further second homology arm have sequences that hybridize to respective sequences on opposite sides of a cut site for the Cas9 protein on the further target genomic sequence.

In embodiments, the invention provides that the second plasmid comprises an allelic Pro-AG system further comprising, (i) a further gRNA having a further sequence that hybridizes to a further target genomic sequence on the target plasmid in the bacteria, wherein the further guide RNA is adjacent to the gRNA outside the gRNA cassette; and (ii) a further first homology arm adjacent to and a further second homology arm outside the gRNA cassette, wherein the further first homology arm and the further second homology arm have sequences that hybridize to respective sequences on opposite sides of a cut site for the Cas9 protein on the further target genomic sequence.

In embodiments, the invention provides a bacterial plasmid gene with a prokaryotic-active genetics (Pro-AG) system comprising: (a) a first plasmid encoding an inducible Cas9 protein; and (b) a second plasmid encoding: (i) a guide ribonucleic acid (gRNA) cassette comprising a promoter for constitutive expression of a gRNA having a sequence that hybridizes to a target genomic sequence on a target plasmid in the bacteria; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iv) an inducible λRed DNA repair cassette. In embodiments, the invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

In embodiments, the invention provides that the gene confers antibiotic resistance. In embodiments, the invention provides that the gene encodes a virulence factor, a membrane transporters or an efflux pump.

In embodiments, the invention provides a bacterial gene editing composition comprising a prokaryotic-active genetics (Pro-AG) system comprising: (a) a first plasmid encoding an inducible Cas9 protein; and (b) a second plasmid encoding: (i) a guide ribonucleic acid (gRNA) cassette comprising a promoter for constitutive expression of a gRNA having a sequence that hybridizes to a target genomic sequence on a target plasmid in the bacteria; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iv) an inducible λRed DNA repair cassette. In embodiments, the invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

In embodiments, the invention provides that the first plasmid further comprises a second guide RNA sequence, wherein the second guide RNA sequence targets a second target genomic sequence. In embodiments, the invention provides that the second target genomic sequence is on the first plasmid, wherein the second guide RNA sequence is flanked by a third homology arm and a fourth homology arm on the first plasmid, and wherein the third homology arm and the fourth homology arm flank the second target genomic sequence on the target plasmid.

In embodiments, the invention provides a Pro-AG dependent genetic relay and switch circuit comprising: a second target plasmid, wherein the second target genomic sequence is on the second target plasmid, wherein the first target plasmid comprises a third homology arm and a fourth homology arm, and when induced the third homology arm and the fourth homology arm flank the second target genomic sequence on the second target plasmid. In embodiments, the invention provides that the first and second guide RNA sequences are first inserted into the first target plasmid and are then inserted into the second target plasmid.

In embodiments, the invention provides a Pro-AG amplifier system comprising an operator/promoter cargo in the gRNA cassette.

In embodiments, the invention provides a method of treating a subject for antibiotic resistant bacteria comprising administering to the subject an effective amount of a pro-active genetics system targeted to an antibiotic resistance gene in the bacteria, as described herein.

### BRIEF DESCRIPTION OF THE DRAWINGS

FIGS. 1a-1d show that prokaryotic-active genetics (Pro-AG) is ˜100× more efficient than CRISPR-control for targeting antibiotic resistance conferred by a high copy plasmid. Schematic of CRISPR-control- (FIG. 1a) and Pro-AG- (FIG. 1c) mediated editing of beta-lactamase gene (bla) encoded on a high-copy plasmid (pET) conferring resistance to ampicillin (Amp^{R}, tan greyscale arrow) in E. coli MG1655. gRNAs were initially expressed from a low copy plasmid (pCRISPR Amp) maintained under spectinomycin (Spm) selection and using the constitutive tet promoter to express the gRNA. A second low copy plasmid, pCas9, maintained under chloramphenicol (Cm^{R}) selection, encodes Cas9 under control of an anhydrotetracycline (aTc) inducible promoter. FIG. 1a shows CRISPR-control configuration: two gRNAs (gRNA1 and gRNA2 carried on the pCRISPR Amp plasmid) were tested for Cas9-mediated targeting at two different locations in the bla gene. The Cas9/gRNA1 and Cas9/gRNA2 induced cleavage sites, and the protospacer adjacent motif (PAM) are indicated as well as a gene cassette carrying the λRed enzymes. FIG. 1b shows recovery of Amp^{R} colony forming units (Log₁₀ CFU/ml) following CRISPR-mediated targeting of the bla gene with gRNAs 1 or 2 in the absence (−aTc, black dots) or presence (+aTc, blue greyscale dots) of aTc-induced Cas9 expression. FIG. 1c shows Pro-AG configuration: the gRNA2 expression cassette flanked by bla (Amp^{R}) homology arms (HA1 and HA2) that directly abut the gRNA2 cleavage site was incorporated into pCRISPR Amp. The Cas9/gRNA2 induced cleavage site, the protospacer adjacent motif (PAM), and the HA1 (dark yellow greyscale box), and HA2 (light yellow greyscale box) homology arms are indicated. Also carried on plasmids pCRISPR Amp and pPro-AG Amp are the recombinogenic λRed enzymes (λRed), which can be induced with L-arabinose (arab). FIG. 1d shows recovery of CFU on Amp plates following CRISPR-control versus Pro-AG-mediated targeting of the bla gene. In this and subsequent Figures, abbreviations for Cas9 induction are as in FIG. 1b; induction of λRed enzymes: (+aTc+arab: red greyscale dots); CRISPR (green greyscale shaded box) and Pro-AG (blue greyscale shaded box) treatments are highlighted. Plasmids sequenced from Amp^{R} colonies (CRISPR, green greyscale box and Pro-AG, blue greyscale box) displayed unaltered gRNA target sites in all clones analyzed (30/30). Data in FIGS. 1b and 1d are plotted as the mean±SEM, representing three independent experiments performed in triplicate and analyzed by Student's t-test. N.S.=not significant (P>0.05) *=P<0.05; **=P<0.01; ****P<0.0001.

FIGS. 2a-2c show efficient reduction of Amp^{R} CFU by Pro-AG results from homology-mediated insertion of active gRNA cassettes into the bla target gene. FIG. 2a shows selection for E. coli CFU on ampicillin (Amp, filled dots) or gentamicin (Gm, open dots) plates following CRISPR-control- or Pro-AG-mediated targeting of the dual antibiotic resistant target plasmid pETg (see pETg plasmid schematic in FIG. 1c from single colonies grown in the presence (+aTc, blue greyscale dots), in the absence (−aTc, black dots) of anhydrotetracycline for induction of Cas9, or in combination of aTc and arabinose (+aTc+arab, red greyscale dots) for induction of Cas9 and λRed, respectively. FIG. 2b shows individual colonies isolated from gentamicin plates following Cas9 and λRed induction under CRISPR (green greyscale shaded box) or Pro-AG (blue greyscale shaded box) regimens in FIG. 2a were struck on new ampicillin (Amp^{R}, left images) and gentamicin (Gm^{R}, right images) plates. Representative images of 200 colonies struck from CRISPR-control (top) or Pro-AG (bottom) are shown. FIG. 2c shows DNA sequence analysis of plasmids isolated from single colonies in FIG. 2b recovered from either the CRISPR-control regimen (Amp^{R} or Gm^{R} plates; green arrow) or the Pro-AG regimen (Gm^{R} plates; blue greyscale arrow). All 30 CRISPR-derivative clones analyzed revealed a fully intact pETg plasmid (unedited, green greyscale circle), while all 30 Pro-AG-derivative clones analyzed carried a perfect insertion of the homology flanked gRNA2 expression cassette into the bla gene (zoom-in bottom scheme). The gRNA expression cassette is composed of the gRNA scaffold (purple greyscale), 20 bp gRNA targeting sequences (pink greyscale and black), and the constitutive tet promoter (grey). Also indicated are the Cas9 cleavage site, and homology arms (HA1, dark yellow greyscale box and HA2, light yellow greyscale box) that flank the gRNA2 cleavage site in the bla target gene carried on the pETg plasmid. Data in a are plotted as the mean±SEM, representing three independent experiments performed in triplicate and analyzed by Student's t-test. N.S.=not significant (P>0.05) *=P<0.05.

FIGS. 3a-3c show the Pro-AG system mediates efficient editing and cargo delivery dependent on precise flanking of the gRNA cassette by homology arms of the target gene. FIG. 3a shows a schematic of plasmids used to compare performance of CRISPR-control (pCRISPRAmp), Pro-AGFP (pPro-AGFPAmp: gRNA2+GFP within homology arms), and external placement of gRNA2 outside of the homology arms flanked cassette (pgRNA Out-Amp: GFP-only within homology arms, gRNA outside of HA-cassette). FIG. 3b shows recovery of E. coli CFU following CRISPR-control or Pro-AG using the three different plasmid configurations indicated in FIG. 3a and the various induction conditions indicated in the key. FIG. 3c shows sequence analysis of targeted plasmids. pETg plasmids recovered from CRISPR-control-treated colonies (green greyscale circle) all display intact target sequence (“escapers”). Analysis of all Pro-AGFP-recovered pETg plasmids (blue greyscale circle) confirmed precise insertion of the gRNA+GFP cassette at the gRNA cut site, which consists of the full gRNA cassette (scaffold, purple greyscale; gRNA, pink greyscale and black and tet promoter, grey) plus GFP. Although the gRNAOut-targeted pETg configuration (red greyscale circle) resulted in ˜100 fold less efficient targeting of the bla target gene than the Pro-AGFP configuration, all plasmids isolated from colonies selected on Gm plates carried precise insertions of the GFP-only cassette. Data in FIG. 3b are plotted as the mean±SEM, representing three independent experiments performed in triplicate and analyzed by Student's t-test. N.S.=not significant (P>0.05); ***=P<0.001; ****P<0.0001.

FIGS. 4a-4b show CRISPR-control versus Pro-AG-mediated gene-editing. Scheme of method used for editing Amp resistance cassette from multi-copy plasmid through CRISPR-control (FIG. 4a) or Pro-AG (FIG. 4b) editing configurations. Escherichia coli MG1655 cells were transformed with pET, pCas9 and pCRISPR or pPro-AG derivative plasmids in FIGS. 4a and 4b, respectively. Cells were selected on LB plates containing triple antibiotic selection: ampicillin, spectinomycin and chloramphenicol (+Amp+Spm+Cm) in the presence or in the absence of anhydrotetracycline (aTc, Cas9 induction). Following transformation, single colonies were selected for an editing round, where bacteria was grown over night in LB broth in the presence of Amp and under induction of Cas9 with aTc. Additionally, in Pro-AG configuration FIG. 4b shows recombinogenic λRed enzymes were induced with the addition of L-arabinose. Finally, aliquots were diluted and plated for selection and quantification of Amp or Gm resistant colonies in each editing configuration.

FIGS. 5a-5b show Escherichia coli MG1655 colony counts and cell density are not affected after transformation and plating with Cas9 switched off or on. FIG. 5a shows Quantification of Ampicillin resistant E. coli expressing CRISPR-control configuration that mediates Cas9 targeting of bla gene. Colony forming units (CFU) were quantified on ampicillin agar plates in the presence (+aTc, blue greyscale dots) or in the absence (−aTc, black dots) of the Cas9 inducer anhydrotetracycline. FIG. 5b shows optical densities measured at 600 nanometers (O.D 600 nm) from E. coli over-night cultures grown in the presence (+aTc, blue greyscale bars) or in the absence (−aTc, black bars) of anhydrotetracycline after CRISPR-control-mediated editing round AmpgRNA1 or AmpgRNA2 were expressed.

FIGS. 6a-6b show a number of Escherichia coli MG1655 colonies and cell density is not affected after transformation and overnight culture steps, respectively under CRISPR-control or Pro-AG editing configurations. FIG. 6a shows quantification of Ampicillin resistant E. coli after transformation with the three plasmids system for CRISPR-control or Pro-AG editing configurations, that mediate Cas9 targeting of bla gene. Colony forming units (CFU) were quantified on LB ampicillin, spectinomycin and chloramphenicol agar plates and in the presence (+aTc, blue greyscale dots) or in the absence (−aTc, black dots) of the Cas9 inducer anhydrotetracycline. FIG. 6b shows optical densities (O.D.) measured at 600 nanometers from single E. coli colonies over-night cultures grown in the presence (+aTc, blue greyscale bars) or in the absence (−aTc, black bars) of anhydrotetracycline, and in combination of λRed enzymes induction with arabinose and Cas9 with aTc (+aTc +arab, red greyscale bars) after CRISPR-control or Pro-AG mediated editing rounds.

FIGS. 7a-7b show Cas9 and λRed induction with anhydrotetracycline and arabinose, respectively, do not affect Escherichia coli viability. FIG. 7a shows quantification of E. coli colony forming units (CFU) on LB agar plates with (+Amp) or without (−Amp) after CRISPR-control or Pro-AG editing rounds when cells were grown in the presence (blue greyscale dots) or in the absence (black dots) of anhydrotetracycline (aTc) or in combination of aTc and arabinose (red greyscale dots). FIG. 7b shows quantification of E. coli ampicillin resistant colony forming units (CFU) on LB ampicillin agar plates after CRISPR-control or Pro-AG editing rounds when cells were grown in the presence (+, red greyscale dots) or in the absence (−, black dots) of arabinose (arab), and under triple antibiotic selection, corresponding to the three plasmids used in the editing protocols.

FIGS. 8a-8b show RecA is partially involved in the mechanism for E. coli escape from Pro-AG-mediated editing. FIG. 8a shows a comparison of CFU selection from wild type E. coli MG1655 (WT) vs. MG1655 isogenic recA mutant (ΔRecA) strains on ampicillin (Amp, filled dots) or gentamicin (Gm, open dots) plates following Pro-AG-mediated targeting of the dual antibiotic resistant target plasmid pETg, from single colonies grown in the absence (−aTc, black dots), in the presence (+aTc, blue greyscale dots) of anhydrotetracycline for induction of Cas9 only, or in combination with arabinose for induction of both Cas9 and λRed (+aTc+arab, red greyscale dots). FIGS. 8a-8b shows DNA sequence analysis of plasmids isolated from single ΔRecA colonies in FIG. 8a recovered from the Pro-AG regimen (Gm^{R} plates; blue greyscale shaded box). All 12 Pro-AG-derivative clones analyzed carried a perfect insertion of the homology flanked gRNA2 expression cassette into the bla gene (zoom-in bottom scheme). The gRNA expression cassette is composed of the gRNA scaffold (purple greyscale), 20 bp gRNA targeting sequences (pink greyscale and black), and the constitutive tet promoter (grey). Also indicated are the Cas9 cleavage site, the protospacer adjacent motif (PAM), and homology arms (HA1, dark yellow greyscale box and HA2, light yellow greyscale box) that flank the gRNA2 cleavage site in the bla target gene carried on the pETg plasmid. Data in FIG. 8a are plotted as the mean±s.e.m. and analyzed by Student's t-test. **(P<0.01); ****(P<0.0001).

FIGS. 9a-9b show sequence analysis of gRNA donor plasmids from Pro-AG escapers. Schematic of pPro-AG (Amp) FIG. 9a and pPro-AG (lacZ) FIG. 12b plasmids. Homology arms 1 (HA1, orange) and 2 (HA2, yellow) flanking bla FIG. 9a and lacZ FIG. 12b gRNAs, replication origin (pSC101ori, red), tet operator (cyan), gRNA (pink greyscale arrow), gRNA scaffold (turquoise greyscale arrow) and spectinomycin resistant gene (Spm^{R}, grey arrow) are indicated. FIG. 9a shows sequencing analysis of pPro-AG (Amp) plasmids from 20 escaper colonies of Pro-AG editing bla plasmid-encoded shows deletions (red greyscale lines) across the plasmid sequence in more than 50% of the plasmids. The remaining plasmids analyzed show intact wild type sequences (continuous black lines). FIG. 9b shows sequencing analysis of pPro-AG (lacZ) plasmids from 10 escaper colonies of Pro-AG editing chromosomal lacZ gene shows similar pattern of tet operator deletion (red line) and sequence insertion (*). Length of insertion (discontinuous black line) and deletions (discontinuous red line) is indicated (bp). The 20 bp upstream and downstream specific insertions or deletions is also shown from the coding DNA strand (5′-3′).

FIGS. 10a-10b show differences in editing performance between Pro-AG and CRISPR-control configurations are partially dependent on the gRNA levels. FIG. 10a shows a schematic of CRISPR-control and Pro-AG-mediated editing configurations of beta-lactamase gene (bla) encoded on high-copy plasmids (pETg-CRISPR and pETg-Pro-AG) conferring resistance to ampicillin (Amp^{R}, tan greyscale arrow) in E. coli MG1655. CRISPR-control configuration expresses gRNA2 targeting bla gene from the same pETgCRISPR target plasmid. Pro-AG configuration expresses the gRNA2 cassette flanked by bla (Amp^{R}) homology arms (HA1 and HA2) that directly abut the gRNA2 cleavage site from the same pETg-Pro-AG target plasmid. pETg-CRISPR and pETg-Pro-AG plasmids also express a gentamicin resistance marker (Gm^{R}, green greyscale arrow). Cells were also transformed with a low copy plasmid, pCas9, maintained under chloramphenicol (Cm^{R}) selection, encodes Cas9 under control of an anhydrotetracycline (aTc) inducible promoter, and with a second low copy plasmid, pAgRNA, maintained under spectinomycin (Spm) selection, encodes λRed under control of an arabinose (arab) inducible promoter. FIG. 10b shows a top panel. Selection for E. coli CFU on ampicillin (Amp, filled dots) or gentamicin (Gm, open dots) plates following CRISPR-control- or Pro-AG-mediated targeting of the dual antibiotic resistant target plasmids pETg-CRISPR and pETg-Pro-AG, respectively, from single colonies grown in the absence (−aTc, black dots), in the presence (+aTc, blue greyscale dots) of anhydrotetracycline for induction of Cas9 only, or in combination with arabinose for induction of both Cas9 and λRed (+aTc+arab, red greyscale dots). Bottom panel. DNA sequence analysis of plasmids isolated from single colonies in top panel recovered from the Pro-AG regimen (Gm^{R} plates; blue greyscale shaded box). All 10 Pro-AG-derivative clones analyzed carried plasmids target with a perfect insertion of the homology flanked gRNA2 expression cassette into the bla gene (zoom-in bottom scheme). The gRNA expression cassette is composed of the gRNA scaffold (purple greyscale), 20 bp gRNA targeting sequences (pink greyscale and black), and the constitutive tet promoter (grey). Also indicated are the Cas9 cleavage site, the protospacer adjacent motif (PAM), and homology arms (HA1, dark yellow greyscale box and HA2, light yellow greyscale box) that flank the gRNA2 cleavage site in the bla target gene carried on the pETg-Pro-AG plasmid. Data in FIG. 10b are plotted as the mean±s.e.m and represent three independent experiments. Data analyzed by Student's t-test. N.S, not significant (P>0.05); ***(P<0.001); ****(P<0.0001).

FIGS. 11a-11b show Pro-AG acts via a self-amplifying mechanism with a copied gRNA that remains functional on further editing events. FIG. 10a shows recovery of spectinomycin resistant (Spm^{R}) E. coli CFU from single CRISPR-control or Pro-AG edited colonies and following growth at 30° C. (black dots) or 37° C. (white dots). FIG. 10b shows a selection for E. coli CFU on ampicillin plates from single CRISPR-control or Pro-AG edited colonies and following growth at 30° C. (filled dots) or 37° C. (open dots) in the absence (−aTc, black dots) or in the presence of anhydrotetracycline in combination with arabinose (+aTc+arab, red greyscale dots). Bacteria growth at 30° C. and 37° C. conditions are permissive and non-permissive for gRNA-harboring plasmids replication, respectively. Data in FIG. 10b are plotted as the mean±s.e.m and analyzed by Student's t-test. N.S, not significant (P>0.05); ***(P<0.001); ****(P<0.0001).

FIGS. 12a-12b show Pro-AG configuration is well suited for manipulation of large plasmids. FIG. 12a shows a schematic of CRISPR-control and Pro-AG-mediated editing configurations of beta-lactamase gene (bla) encoded on long cosmid vector (Super-Cos SV3B05) conferring resistance to ampicillin (Amp^{R}, tan greyscale arrow) in E. coli MG1655. Super-Cos SV3B05 cosmid also expresses a kanamycin resistance marker (Km^{R}, green arrow). Cells were also transformed with a low copy plasmid, pCas9, maintained under chloramphenicol (Cm^{R}) selection, encodes Cas9 under control of an anhydrotetracycline (aTc) inducible promoter. CRISPR control configuration expresses gRNA2 targeting bla gene from pCRISPR Amp plasmid. Pro-AG configuration expresses the gRNA2 cassette flanked by bla (Amp^{R}) homology arms (HA1 and HA2) that directly abut the gRNA2 cleavage site from pPro-AG Super-Cos plasmid. Also carried on plasmids pCRISPR Amp and pPro-AG Super-Cos are the recombinogenic λRed enzymes (λRed), which can be induced with L-arabinose (arab). FIG. 12b shows a top panel. Selection for E. coli CFU on ampicillin (Amp, filled dots) or kanamycin (km, open dots) plates following CRISPR-control- or Pro-AG-mediated targeting of the dual antibiotic resistant target cosmid Super-Cos SV3B05, from single colonies grown in the absence (−aTc, black dots), in the presence (+aTc, blue greyscale dots) of anhydrotetracycline for induction of Cas9 only, or in combination with arabinose for induction of Cas9 and λRed (+aTc+arab, red greyscale dots). FIG. 12b shows a bottom panel. DNA sequence analysis of plasmids isolated from single colonies in top panel recovered from the Pro-AG regimen (Km^{R} plates; blue greyscale shaded box). All 10 Pro-AG-derivative clones analyzed carried a perfect insertion of the homology flanked gRNA2 expression cassette into the bla gene (zoom-in bottom scheme). The gRNA expression cassette is composed of the gRNA scaffold (purple greyscale), 20 bp gRNA targeting sequences (pink greyscale and black), and the constitutive tet promoter (grey). Also indicated are the Cas9 cleavage site, the protospacer adjacent motif (PAM), and homology arms (HA1, dark yellow greyscale box and HA2, light yellow greyscale box) that flank the gRNA2 cleavage site in the bla target gene carried on the Super-Cos SV3B05 cosmid. Data in FIG. 12b are plotted as the mean±s.e.m and analyzed by Student's t-test. N.S, not significant (P>0.05); ****(P<0.0001).

FIGS. 13a-13e show a comparison between CRISPR-control and Pro-AG editing configurations when targeting chromosomal Escherichia coli lacZ gene. FIG. 13a shows a schematic of the three plasmids used for editing the genomic lacZ locus: CRISPR-control (pCRISPRlacZ); gRNA-only or Pro-AG (pPro-AGlacZ); and gRNA+GFP or Pro-AGFP (pPro-AGFPlacZ). Precise insertion of homology flanked cassettes would inactivate the lacZ chromosomal E. coli locus (lacZ, blue greyscale arrow) and in the case of the pPro-AGFPlacZ construct, result in in-frame fusion with GFP. FIG. 13b shows quantification of E. coli colony forming units (CFU) following initial transformation with the corresponding CRISPR-control or Pro-AG editing configuration plasmids. CFUs were quantified on LB spectinomycin and chloramphenicol agar plates and in the presence (+aTc, blue greyscale dots) or in the absence (−aTc, black dots) of the Cas9 inducer anhydrotetracycline. FIG. 13c shows optical densities measured at 600 nanometers (O.D 600 nm) from single E. coli colonies after transformation and over night cultures when cells were grown in the absence (−aTc, black bars), or presence of anhydrotetracycline (+aTc, blue greyscale bars) or in combination of aTc and arabinose (+aTc+arab, red greyscale bars) compared under the following configurations: CRISPR, Pro-AG, or Pro-AGFP. FIG. 13d shows quantification of E. coli colony forming units (CFU) after CRISPR-control, Pro-AG and Pro-AGFP mediated editing of lacZ loci. Cells were grown with (+aTc, blue greyscale dots) or without (−aTc, black dots) Cas9 induction with anhydrotetracycline, or in combination of Cas9 and λRed induction with aTc and arabinose, respectively (+aTc+arab, red greyscale dots). FIG. 13e shows appearance of dilutions spots carrying isolated colonies obtained under CRISPR-control (top) versus Pro-AG (bottom) configurations and after induction of Cas9 only. Data in FIGS. 13b, 13c, and 13d are plotted as the mean±s.e.m and represent three independent experiments. Data analyzed by Student's t-test. **(P<0.01); ****(P<0.0001).

FIGS. 14a-14d shows the Pro-AG configuration mediates enhanced editing of chromosomal targets. FIG. 14a shows quantification of E. coli lacZ loci editing or mutagenic events (% white colonies of total colonies recovered), on plates-containing IPTG and X-Gal and after treatments with CRISPR-control, Pro-AG, or Pro-AGFP systems (top panel). Representative images of IPTG/X-Gal plates containing unedited (blue colonies) or edited (white colonies) are shown for the corresponding editing configuration (bottom panel). FIG. 14b shows sequence analysis of edited lacZ genomic targets. CRISPR-control edited colonies displayed large chromosomal deletions (*) of the lacZ locus (top scheme), which were not analyzed further. Pro-AG events all resulted in the precise insertion of full lacZ-gRNA cassette within the gRNA target site in lacZ (middle scheme), and all Pro-AGFP events contained perfect insertions of the gRNA+GFP cassette (bottom scheme) resulting in in-frame fusion of the lacZ and gfp genes. FIG. 14c shows fluorescence microscopy images from E. coli over-night cultures expressing Pro-AGFP configuration and growing under Cas9 only (left panels, aTc+arab−), or in combination of Cas9 and λRed (right panels, aTc+ arab+) activation conditions (+aTc+arab) and in the presence of the lac operon inducer molecule IPTG. GFP (bottom panels, green) and merged fluorescence and phase-contrast images (top panels) are shown. Scale bars (20 μm). FIG. 14d shows quantification of GFP fluorescence from E. coli single colonies after Pro-AGFP configuration treatment in the absence (−) or in the presence (+) of Cas9 (aTc) and λRed (arab) activation conditions. The Y-axis quantifies the ratio of absorbance at 525 nm/600 nm and is indicated as GFP fluorescence/OD in relative units (RU). Data in FIGS. 14a and 14d were plotted as the mean±s.e.m and represent five independent experiments and 50 single colonies from each condition, respectively. Data analyzed by Student's t-test. N.S, not significant (P>0.05); ****(P<0.0001).

FIGS. 15a-15d show effect of gRNA placement on targeting the chromosomal lacZ gene. FIG. 15a shows schematic of plasmids used to compare performance of Pro-AGFP (pPro-AGFP lacZ: lacZgRNA+GFP within homology arms), and external placement of lacZgRNA outside of the homology arms flanked cassette (pgRNAOut-lacZ; GFP-only within homology arms, gRNA outside of HA-cassette). FIG. 15b shows quantification of E. coli colony forming units (CFU) after, Pro-AGFP and gRNA-Out mediated editing of lacZ loci. Cells were grown with (+aTc, blue greyscale dots) or without (−aTc, black dots) Cas9 induction with anhydrotetracycline, or in combination of Cas9 and λRed induction with aTc and arabinose, respectively (+aTc +arab, red greyscale dots). FIG. 15c shows quantification of E. coli lacZ loci editing or mutagenic events (% white colonies of total colonies recovered), on plates-containing IPTG and X-Gal and after treatments with Pro-AGFP and gRNA-Out configurations (top panel). Representative images of IPTG/X-Gal plates containing unedited (blue colonies) or edited (white colonies) are shown for the corresponding editing configuration (bottom panel). FIG. 15d shows sequence analysis of edited lacZ genomic targets. All Pro-AGFP events contained perfect insertions of the gRNA+GFP cassette (top scheme). All gRNA-Out events contained perfect insertions of the GFP-only cassette (bottom scheme). Data in FIG. 15b are plotted as the mean±s.e.m and analyzed by Student's t-test. N.S, not significant (P>0.05); ****(P<0.0001).

FIGS. 16a-16c show pro-Active Genetic (Pro-AG) systems in E. coli. FIG. 16a is a diagram of Pro-AG system components. Low copy-number plasmid-A (CmR) expresses Cas9 upon induction with anhydrotetracycline (aTC); low copy-number plasmid-BC (SpmR) expresses a gRNA constitutively (gray promoter box), which is either flanked (Pro-AG) or not (Control CRISPR) by homology arms (HA1, HA2), and also carries a λRed DNA repair cassette, inducible by arabinose (arab, red dots); and high-copy-number target plasmid pET (AmpR, GmR). beta lactamase (bla=AmpR) is targeted for gRNA directed cleavage. FIG. 16b shows bacteria carrying three “Control CRISPR” or “Pro-AG” plasmids were grown overnight±aTC (Cas9 inducer), antibiotics for plasmid retention, and were inoculated onto Amp or Gm plates. Control CRISPR reduced CFU recovered on either Amp or Gm plates upon Cas9 induction by ˜100-fold, while Pro-AG reduced CFU recovered by ˜100,000-fold on Amp plates, which could be quantitively recovered on Gm plates. FIG. 16c is a diagram illustrating precise editing of the bla locus by Pro-AG resulting in precise insertion of the gRNA transgene into its target site. Burgundy box indicates the PAM site present in the bla target gene; orange box: the first three bases of the gRNA recognition site abutting the cut site: the gRNA scaffold: the 5′most 17 bp of the gRNA recognition sequence: the Tet promoter driving constitutive gRNA expression.

FIGS. 17a-17d show that Pro-Active requires gRNA amplification. FIGS. 17a-17c are diagrams of tested Pro-AG plasmid-B,C constructs: Control CRISPR (gRNA-bla alone) (FIG. 17a); B) Pro-A•GFP In: Pro-AG configuration for gRNA+gfp cargo (gRNA-bla and gfp flanked by homology arms: HA1,HA2) (FIG. 17b); C) Pro-AGFP Out: gRNA in control CRISPR configuration and only gfp flanked by HA (FIG. 17c). D) CRISPR targeting of bla gene in pET plasmid (FIG. 17d) (see also FIG. 16a). Y-axis: CFU recovered on Amp or Gm plates following induction of Cas9 expression with aTC from plasmid-A±arab induction of λRed DNA repair cassette from plasmid-B,C.

FIGS. 18a-18 show Dual Pro-Ag systems. FIG. 18a shows a dual gRNA cassette plasmid-BC Pro-AG system consists of: a <gRNA-bla> cassette flanked by homology arms (HA1b,HA2b) identical to that shown in FIG. 16a, and a second <gfp,gRNA-lacZ> cassette carrying a gfp transgene flanked by homology arms Ha1z,HA2z. gfp coding sequences are in-frame with the adjacent HA2z encoded bla sequences such that when integrated into a full bla gene, a fluorescent Bla-GFP fusion protein is produced (see FIG. 18b). Following standard Pro-AG protocols as in FIG. 16a (i.e., induction of Cas9 by aTC, not shown here, and λRed by arab), the gRNA-bla cassette inserts into the bla target gene carried by either high- (pET, FIG. 18e, top plasmid) or medium-copy number (pSV3B05-lacZ, FIG. 18e, bottom plasmid) plasmids. The <gfp, gRNA-lacZ> cassette either inserts into a single-copy genomic locus (FIG. 18b), when gRNA-bla targets pET, or into medium-copy number pSV3B05 (FIG. 18e, bottom plasmid), which also carries a bla target gene. FIG. 18b shows a conditionally (±IPTG) expressed genomic lacZ target gene. Pro-AG editing inserts the <gfp,gRNA-lacZ> cassette into the gRNA-lacZ cleavage site, IPTG induces LacZ-GFP fusion protein. FIG. 18c is a nested arrangement: HA outer arms (HAz1,HAz2) flank both gfp,gRNA-lacZ and the <gRNA-bla> cassette. FIG. 18d shows Pro-AG editing inserts the compound gfp,gRNA-lacZ,<gRNA-bla> cassette into genomic lacZ target. FIG. 18e shows the target plasmids pET (high copy number, FIG. 18e, top plasmid) and pSV3B05 (medium copy number, FIG. 18e, bottom plasmid). FIG. 18f shows Pro-AG allelic editing: plasmid-BC carries both gRNA-bla and gRNA-lacZ between arms HA1b and HA2b, and also a separate editing template (HA2z/HA1Z) to repair mutant lacZ alleles (asterisks in FIG. 18g) that can be cut by gRNA-lacZ (the repair template lacking the gRNA cleavage site cannot be cut by gRNA-lacZ). In analogy to allelic-drive demonstrated in fruit flies, Pro-AG editing repairs a mutant allele that either lies within the gRNA recognition sequence (Copy-Cutting) or just adjacent to it (Copy-Grafting) (FIG. 18g). In either case, the gRNA cuts only the mutant allele.

FIGS. 19a-19e show a Pro-AG relay system. In FIG. 19a, a donor plasmid-B,C carries a gfp transgene with a minus-1 frameshift mutation (gfp-1) and two gRNAs targeting the bla gene, the first (gRNA1) cuts at the same site as in other experiments, and the second (gRNA2) can only cut an artificial site created by joining sequences brought together by a deletion encompassing HA1 and HA2, which results in fusing the HA3 and HA4 sequences of bla (FIG. 19d). FIG. 19b shows the standard pET target plasmid with primary (HA1,HA2), and secondary (HA3,HA4) homology arms labeled as well as the PAM sites recognized by gRNA1 (PAM-1) and gRNA2 (PAM-2). FIG. 19c shows insertion of the gRNA relay cassette into the primary pET gRNA-directed cut site. Note that the <gRNA2,gRNA1,gfp-1> cassette in pET•gfp-1 is now also flanked by HA3 and HA4. FIG. 19d shows synthetic genomic target site for gRNA2 consisting of a bla transgene deleted for HA1 and HA2 sequences. The bla transgene also carries a plus-1 frameshift at in HA4 but does not encode a termination codon until after its junction with HA3. FIG. 19e shows final edited product of relay system with the <gRNA2,gRNA1,gfp-1> cassette inserted into the genomic target site. The combination of the plus-1 frameshift in bla and minus-1 frameshift in gfp result in a restored reading frame and functional expression of GFP.

FIGS. 20a-20b show Pro-AG amplifier systems: cis and trans. Donor plasmid-B,C “cis” carries the mazEF 77 bp operator/promoter as cargo in the <mazEF O/P gRNA-bla> cassette (FIG. 20a). By copying this cassette into low (genome=1 copy), medium (pSV3B05=10-15 copies), or high (pET=˜50 copies) copy number targets differing levels of titration of the MazEF repressor should result in a threshold-dependent activation of both the endogenous MazEF operon and the MazEF-GFP reporter gene (inserted either into the genome or a plasmid (e.g., pET or pSV3B05) to vary its gene dosage. Donor plasmid-B,C “cis” carries a FIS transgene under control of the IPTG inducible lac promoter (FIG. 20b). FIS protein binds to site F in the mazEF promoter to activate transcription. Differing levels of FIS should then result in threshold dependent activation of both the endogenous MazEF operon and the MazEF-GFP reporter.

FIGS. 21a-21d show Conjugal and phage Pro-AG systems. Pro-AG components—plasmid-A: aTC inducible cas9; plasmid-BC: HA flanked <gRNA >+λRed cassettes (FIG. 21a). Pro-MobV: Pro-AG components from plasmid-A (cas9±aTC)+plasmid-B,C (<gRNA>λRed±arab)+conjugation machinery (e.g., TSS4 system) and cis-acting oriT components of the conjugal plasmid pNuc-cis [1]+two AR selectable markers (Cm^{R}, Spm^{R})+HA-tagged gfp⁻¹ marker. bla gene targets in both high (pET) and medium (pSV3B05) copy number plasmids (FIG. 21c). Recipient cells are marked by lacZ⁻, mCherry⁺. phage λPro-AG system. A nonessential region of the λ-phage genome will be excised (19,014-27,480 bp) and replaced (FIG. 21d). Genes in this deleted region are not repressed by λ cI and can be activated in dormant lysogens. λPro-AG will target Amp^{R} plasmids (FIG. 21c) using gRNA-bla.

FIGS. 22a-22b show eukaryotic gene-drive systems. FIG. 22a shows the bipartite synthetic CRISPR system. A gRNA binds Cas9 directing it to bind and cleave DNA at complementary sites 20 nucleotides in length. The PAM site (NGG), required for Cas9 binding to genomic targets, is absent in the gRNA. In FIG. 22b a Cas9+gRNA cassette inserted in one chromosome directs cleavage of its homolog in the germline and is copied into the DNA break by homology directed DNA repair (HDR) resulting in nearly all progeny (˜99%) inheriting the “gene-drive” cassette.

### DETAILED DESCRIPTION

This application incorporates by reference herein the entirety of PCT/US2016/052424 published as WO2017/049266, which describes a variety of genetic editing constructs that can be used in the present invention.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The disclosure provides compositions, systems and methods for reducing antibiotic resistance in bacteria comprising modifying a bacterial genome with a pro-active genetics system.

In embodiments, the prokaryotic-active genetics system comprises a method of inhibiting antibiotic resistance in bacteria comprising modifying a bacterial plasmid gene for antibiotic resistance with a prokaryotic-active genetics (Pro-AG) system. In embodiments, the Pro-AG system comprises a first plasmid encoding an inducible Cas9 protein. In embodiments, the Pro-AG system further comprises a second plasmid encoding: (i) a guide ribonucleic acid (gRNA) cassette comprising a promoter for constitutive expression of a gRNA having a sequence that hybridizes to a target genomic sequence on a target plasmid in the bacteria, wherein the target genomic sequence in the bacteria confers antibiotic resistance; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iii) an inducible λRed DNA repair cassette. The invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby modifying the bacterial plasmid gene for antibiotic resistance. The invention provides that other DNA repair systems may be employed in the DNA repair cassette besides λRed.

In embodiments, the invention provides that copies of the gRNA cassette increase via a positive feedback loop allowing for self-amplification of the Pro-AG system. In embodiments, the invention provides that the first plasmid is present in a lower copy number than the second plasmid and the bacterial plasmid. In embodiments, the invention provides that the bacterial gene for antibiotic resistance is a beta-lactamase gene, however, any gene encoding for resistance to any antibiotic is contemplated by the invention. In embodiments, the invention provides that the antibiotic is ampicillin or gentamicin. In embodiments, the invention provides that the bacteria is Escherichia coli, however, any bacterial organism or cell having plasmid DNA is contemplated by the invention.

In embodiments, the invention provides that the Cas9 protein is induced with anhydrotetracycline. In embodiments, the invention provides that the λRed DNA repair cassette is induced with arabinose. The invention contemplates the use of any induction system. In embodiments, the invention provides that a Tet promoter drives constitutive expression of the gRNA, however, any promoter system is contemplated by the invention.

In embodiments, the invention provides that the bacteria is in a subject, such as a human or other animal. In embodiments, the invention provides that the bacteria is on a solid surface or in a liquid. In embodiments, the invention provides that the bacteria is in a plant or other living organism.

In embodiments, the invention provides that the second plasmid further comprises at least one cargo sequence, which is inserted into the bacterial plasmid. In embodiments, the invention provides that the at least one cargo sequence encodes GFP, or other detectablemarker. In embodiments, the invention provides that the at least one cargo sequence is not flanked by the first and second homology sequences on the second plasmid.

In embodiments, the invention provides that the second plasmid comprises a dual Pro-AG system further comprising, (i) a further guide ribonucleic acid (gRNA) cassette comprising a further promoter for constitutive expression of a further gRNA having a further sequence that hybridizes to a further target genomic sequence on the target plasmid in the bacteria; and (ii) a further first homology arm and a further second homology arm each flanking opposite ends of the further gRNA cassette in the second plasmid, wherein the further first homology arm and the further second homology arm have sequences that hybridize to respective sequences on opposite sides of a cut site for the Cas9 protein on the further target genomic sequence.

In embodiments, the invention provides that the second plasmid comprises a nested Pro-AG system further comprising, (i) a further gRNA having a further sequence that hybridizes to a further target genomic sequence on the target plasmid in the bacteria, wherein the further guide RNA is adjacent to the first homology arm outside the gRNA cassette; and (ii) a further first homology arm outside the gRNA cassette on a side of the plasmid adjacent the second homology arm, and a further second homology arm outside the gRNA cassette on an opposite side of the plasmid adjacent the further gRNA, wherein the further first homology arm and the further second homology arm have sequences that hybridize to respective sequences on opposite sides of a cut site for the Cas9 protein on the further target genomic sequence.

In embodiments, the invention provides that the second plasmid comprises an allelic Pro-AG system further comprising, (i) a further gRNA having a further sequence that hybridizes to a further target genomic sequence on the target plasmid in the bacteria, wherein the further guide RNA is adjacent to the gRNA outside the gRNA cassette; and (ii) a further first homology arm adjacent to and a further second homology arm outside the gRNA cassette, wherein the further first homology arm and the further second homology arm have sequences that hybridize to respective sequences on opposite sides of a cut site for the Cas9 protein on the further target genomic sequence.

In embodiments, the invention provides a bacterial plasmid gene with a prokaryotic-active genetics (Pro-AG) system comprising: (a) a first plasmid encoding an inducible Cas9 protein; and (b) a second plasmid encoding: (i) a guide ribonucleic acid (gRNA) cassette comprising a promoter for constitutive expression of a gRNA having a sequence that hybridizes to a target genomic sequence on a target plasmid in the bacteria; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iv) an inducible λRed DNA repair cassette. In embodiments, the invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

In embodiments, the invention provides that the gene confers antibiotic resistance. In embodiments, the invention provides that the gene encodes a virulence factor, a membrane transporters or an efflux pump.

In embodiments, the invention provides a bacterial gene editing composition comprising a prokaryotic-active genetics (Pro-AG) system comprising: (a) a first plasmid encoding an inducible Cas9 protein; and (b) a second plasmid encoding: (i) a guide ribonucleic acid (gRNA) cassette comprising a promoter for constitutive expression of a gRNA having a sequence that hybridizes to a target genomic sequence on a target plasmid in the bacteria; (ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and (iv) an inducible λRed DNA repair cassette. In embodiments, the invention provides that inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

In embodiments, the invention provides that the first plasmid further comprises a second guide RNA sequence, wherein the second guide RNA sequence targets a second target genomic sequence. In embodiments, the invention provides that the second target genomic sequence is on the first plasmid, wherein the second guide RNA sequence is flanked by a third homology arm and a fourth homology arm on the first plasmid, and wherein the third homology arm and the fourth homology arm flank the second target genomic sequence on the target plasmid.

In embodiments, the invention provides a Pro-AG dependent genetic relay and switch circuit comprising: a second target plasmid, wherein the second target genomic sequence is on the second target plasmid, wherein the first target plasmid comprises a third homology arm and a fourth homology arm, and when induced the third homology arm and the fourth homology arm flank the second target genomic sequence on the second target plasmid. In embodiments, the invention provides that the first and second guide RNA sequences are first inserted into the first target plasmid and are then inserted into the second target plasmid.

In embodiments, the invention provides a Pro-AG amplifier system comprising an operator/promoter cargo in the gRNA cassette.

In embodiments, the invention provides a method of treating a subject for antibiotic resistant bacteria comprising administering to the subject an effective amount of a pro-active genetics system targeted to an antibiotic resistance gene in the bacteria, as described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a genetic construct, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “prokaryote” or “prokaryotic” refers to a cellular organism including those in the genus Bacterium, commonly known as bacteria.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one unit form, or a kit of parts for the combined administration where one or more active compounds and a combination partner (e.g., a pro-active genetic system/constructs plus another drug as explained below, such as an antibiotic agent also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with antibiotic resistance in bacteria. For example, an effective amount in reference to diseases is that amount which is sufficient to improve susceptibility of the bacteria to treatment with antibiotics; or if disease pathology has begun, to palliate, ameliorate, stabilize, reverse or slow progression of the bacterial infection-associated disease, or otherwise reduce pathological consequences of the disease. In any case, an effective amount may be given in single or divided doses.

As used herein, the terms “reduce,” “reducing,” “inhibit” or “inhibiting” with respect to antibiotic resistance embraces at least an amelioration of the ability of an organism, e.g. a bacteria, to not respond to an antibiotic administration, whereby the bacteria is thus made more susceptible to inactivity or death upon an antibiotic administration than before the bacterial gene editing as described herein.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the bacteria, disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of bacterial infection, disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Complementary” or “complement thereof” means that a contiguous nucleic acid base sequence is capable of hybridizing to another base sequence by standard base pairing (hydrogen bonding) between a series of complementary bases.

Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) ′“Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

A nucleic acid is typically single-stranded or double-stranded and will generally contain phosphodiester bonds, although in some cases, as outlined, herein, nucleic acid analogs are included that may have alternate backbones, including, for example and without limitation, phosphoramide (Beaucage et al. (1993) Tetrahedron 49(10):1925 and references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26: 1419, which are each incorporated by reference), phosphorothioate (Mag et al. (1991) Nucleic Acids Res. 19:1437; and U.S. Pat. No. 5,644,048, which are both incorporated by reference), phosphorodithioate (Briu et al. (1989) J. Am. Chem. Soc. 111:2321, which is incorporated by reference), O-methylphosphoroamidite linkages (see Eckstein, Oligonucleotides and Analogues: A Practical Approach, Oxford University Press (1992), which is incorporated by reference), and peptide nucleic acid backbones and linkages (see, Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207, which are each incorporated by reference). Other analog nucleic acids include those with positively charged backbones (Denpcy et al. (1995) Proc. Natl. Acad. Sci. USA 92:6097, which is incorporated by reference); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem: Lett. 4: 395; Jeffs et al. (1994) J. Biomolecular NMR 34:17; and Tetrahedron Lett. 37:743 (1996), which are each incorporated by reference) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (see Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labels, or to alter the stability and half-life of such molecules in physiological environments.

In addition to these naturally occurring heterocyclic bases that are typically found in nucleic acids (e.g., adenine, guanine, thymine, cytosine, and uracil), nucleic acid analogs also include those having non-naturally occurring heterocyclic or modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (TO modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

An “oligonucleotide” or “oligomer” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, or other methods known in the art. All of these references are incorporated by reference.

The elements of a CRISPR-Cas system include a guide RNA, e.g., a gRNA also referred to as crRNA, and a Cas protein. The crRNA or the derivative thereof contains a target specific nucleotide region complementary or substantially complementary to a region of the target nucleic acid. In some embodiments, the crRNA or the derivative thereof contains a user-selectable RNA sequence that permits specific targeting of the enzyme to a complementary double-stranded DNA. In some embodiments, the user-selectable RNA sequence contains 20-50 nucleotides complementary or substantially complementary to a region of the target DNA sequence. In some embodiments, the user-selectable RNA sequence contains less than 20 nucleotides complementary or substantially complementary to a region of the target DNA sequence. Exemplary user-selectable RNA sequence contains 1, 2, 3, 4, 5, 6, 7, 8, 9, 10, 11, 12, 13, 14, 15, 16, 17, 18, or 19 nucleotides complementary or substantially complementary to a region of the target DNA sequence. In some embodiments, the target specific nucleotide region of the crRNA has 100% base pair matching with the region of the target nucleic acid. In some embodiments, the target specific nucleotide region of the crRNA has 90%-100%, 80%-100%, or 70%-100% base pair matching with the region of the target nucleic acid. In some embodiments, there is one base pair mismatch between the target specific nucleotide region of the crRNA and the region of the target nucleic acid. In some embodiments, there are two base pair mismatches between the target specific nucleotide region of the crRNA and the region of the target nucleic acid. In some embodiments, there are three base pair mismatches between the target specific nucleotide region of the crRNA and the region of the target nucleic acid. In some embodiments, there are four base pair mismatches between the target specific nucleotide region of the crRNA and the region of the target nucleic acid. In some embodiments, there are five base pair mismatches between the target specific nucleotide region of the crRNA and the region of the target nucleic acid.

In some embodiments, the system provided herein further includes a trans-activating crRNA (tracrRNA) or a derivative thereof.

The CRISPR-Cas systems provided herein include engineered and/or programmed nuclease systems derived from naturally occurring CRISPR-Cas systems. CRISPR-Cas systems may include contain engineered and/or mutated Cas proteins. CRISPR-Cas systems may also contain engineered and/or programmed guide RNA. In some embodiments, the crRNA or the derivative thereof provided herein is a polynucleotide having a crRNA polynucleotide fused to a tracrRNA polynucleotide. A chimeric single-guided RNA (sgRNA) is described in Jinek et al., 2012, Science 337, 816-821, which is incorporated herein in its entirety. In one embodiment, the Cas protein or the variant thereof provided herein can be directed by a chimeric sgRNA to any genomic locus followed by a 5′-NGG protospacer-adjacent motif (PAM). For example, in some embodiments, crRNA and tracrRNA are synthesized by in vitro transcription, using a synthetic double stranded DNA template containing the promoter. The tracrRNA has a fixed sequence, whereas the target sequence dictates part of crRNA's sequence. Equal molarities of crRNA and tracrRNA are mixed and heated at 55° C. for 30 seconds. Cas9 is added at the same molarity at 37° C. and incubated for 10 minutes with the RNA mix. 10-20 fold molar excess of Cas9 complex is then added to the target DNA. The binding reaction can occur within 15 minutes.

In some embodiments, the Cas protein or the variant thereof is a Cas9 protein or a variant thereof. Isolated Cas9-crRNA complex from the S. thermophilus CRISPR-Cas system as well as complex assembled in vitro from separate components demonstrate that it binds to both synthetic oligodeoxynucleotide and plasmid DNA bearing a nucleotide sequence complementary to the crRNA. It has been shown that Cas9 has two nuclease domains—RuvC- and HNH-active sites/nuclease domains, and these two nuclease domains are responsible for the cleavage of opposite DNA strands. In some embodiments, the Cas9 protein is derived from Cas9 protein of S. thermophilus CRISPR-Cas system. In some embodiments, the Cas9 protein is a multi-domain protein having about 1,409 amino acids residues.

It should be appreciated that any CRISPR-Cas systems capable of disrupting the double stranded nucleic acid and creating a loop structure can be used in the present methods. For example, the Cas proteins provided herein may include, but not limited to, the Cas proteins described in Haft et al., PLoS Comput Biol., 2005, 1(6): e60, and Zhang et al., Nucl. Acids Res., 2013, 10.1093/nar/gkt1262. Some these CRISPR-Cas systems require that a specific sequence be present for these CRISPR-Cas systems to recognize and bind to the target sequence. For instance, Cas9 may require the presence of a 5′-NGG protospacer-adjacent motif (PAM). Thus, in some embodiments, a PAM sequence or a sequence complementary to a PAM sequence is engineered into the target nucleic acid for initiating the binding of the CRISPR-Cas systems to the target nucleic acid.

### Examples

The invention provides a Pro-AG gene-drive system for the bacterium Escherichia coli, for example, that efficiently copies a functional gRNA cassette flanked with sequences homologous to the targeted gRNA/Cas9 cleavage site. Pro-AG inactivates an antibiotic resistance marker on a high copy number plasmid with ˜100-fold greater efficiency than control CRISPR-based methods, indicating a self-amplifying positive feedback loop linked to increasing gRNA dosage. Likewise, the system can efficiently edit a large plasmid, target genes on the bacterial chromosome, or be adapted to introduce functional gene cargos alongside the gRNA cassette. Pro-AG expands the available toolkit for engineering or manipulating bacteria in future biotechnology and biomedicine applications.

The discovery CRISPR has led to a revolution in precision genetic engineering in both prokaryotic and eukaryotic organisms. Type II CRISPR systems, the best studied and most widely applied to practical ends, include both protein (e.g., Cas9) and RNA (e.g., guide RNAs=gRNAs), which form ribonucleotide-protein complexes that cut DNA at sites complementary to a 20 base pair target recognition sequence in the gRNA. In prokaryotes, CRISPR has been employed for efficient genome editing (combined with the λRed homology-directed DNA repair cassette), to kill specific bacterial strains (Cas9 cuts strain-specific genomic sequences in bacteria unable to repair double stranded DNA breaks)^{10,24-25}, or to serve as anti-AR systems that deplete plasmid encoded AR genes (by cleaving and destroying the AR target plasmid)^{10, 24-27}. These latter systems for eliminating high copy-number plasmids typically reduce AR prevalence by two to three logs^{10,24,26}.

Two horizontal gene transfer (HGT) systems for disseminating CRISPR-based anti-AR platforms have been developed. The first makes use of either phagemids, which are packaged with helper phage²⁴⁻²⁵, or temperate phage inserted as pro-phage into the bacterial genome²⁷. These HGT systems have modest efficacies in reducing systemic infections¹⁰, eliminating bacteria from exposed surfaces²⁵, and treating bacterial skin infections in mice^{24,27}. Embedding CRISPR machinery in a phage-like pathogenicity island²⁸ also showed promise. The second HGT system relies on conjugal transfer plasmids employing type IV secretory systems (TSS4) to disseminate CRISPR components to Gram-negative²² or Gram-positive bacteria²⁹. The Gram-negative system, developed in E. coli and carrying essential conjugal transfer genes (including a TSS4 cassette) in cis, is transferred with great efficiency to the closely related S. enterica, in which it targeted specific genomic loci with efficient bactericidal outcomes. The Gram-positive targeting system, although effective in reversing AR in laboratory culture conditions, provided only minimal protection in an intestinal infection model, potentially reflecting difficulties in gaining access to isolated foci of pathogenic bacteria.

The Bier Lab developed the first CRISPR-based gene-drive system in sexually reproducing metazoans, which acted dominantly in somatic cells to generate a whole-body pigmentation phenotype, and also within the germline to copy itself in nearly all gametes². Shortly thereafter, in collaboration with Anthony James at UCI, the first CRISPR gene-drive in mosquitoes was developed, which transmitted in a highly efficient “super-Mendelian” fashion to >99% of progeny and carrying a gene cassette conferring resistance to malarial parasites³. Second generation optimized drive systems were developed³⁰. The design of CRISPR gene-drive systems (“full drive systems”) relies on: a cassette encoding Cas9, a gRNA, and optional cargo (such as anti-malarial single chain antibodies) is inserted precisely at the genomic site targeted for Cas9/gRNA-mediated cleavage¹⁷ (FIGS. 22a-22b). Expression of Cas9 and the gRNA in germline cells then leads to cleavage of the homolog chromosome and homologous DNA Repair (HDR) copies the gene-drive element into the DNA break, resulting in nearly all offspring inheriting the element. Because of the bipartite nature of the Cas9/gRNA system, it is also possible to insert only the gRNA component (±cargo), while providing Cas9 from a separate chromosomal location.

In this invention, active genetic self-amplifying systems were developed in prokaryotes.

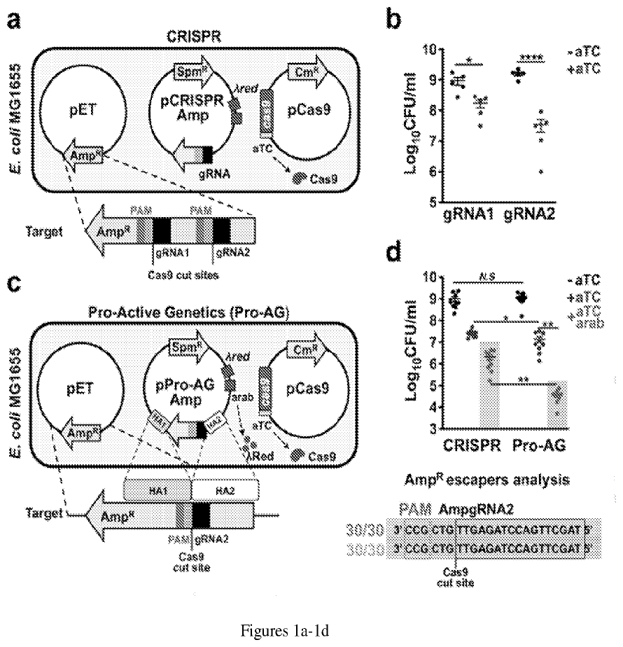

First, a set of three mutually compatible plasmids were employed to assess Cas9-mediated cleavage/inactivation of the beta-lactamase (bla) target gene, which confers ampicillin resistance (Amp^{R}) in Escherichia coli. The three plasmids were: 1) high copy number pET bearing the bla target gene and conferring Amp^{R}; 2) low copy pCRISPRAmp carrying one of two different gRNAs targeting bla sequences under constitutive transcriptional control and maintained under spectinomycin selection (Spm^{R}); and 3) low copy pCas9 expressing Cas9 under control of a tet promoter, inducible with anhydrotetracycline (aTc) and propagated under chloramphenicol selection (Cm^{R}) (FIG. 1a; Table 1).

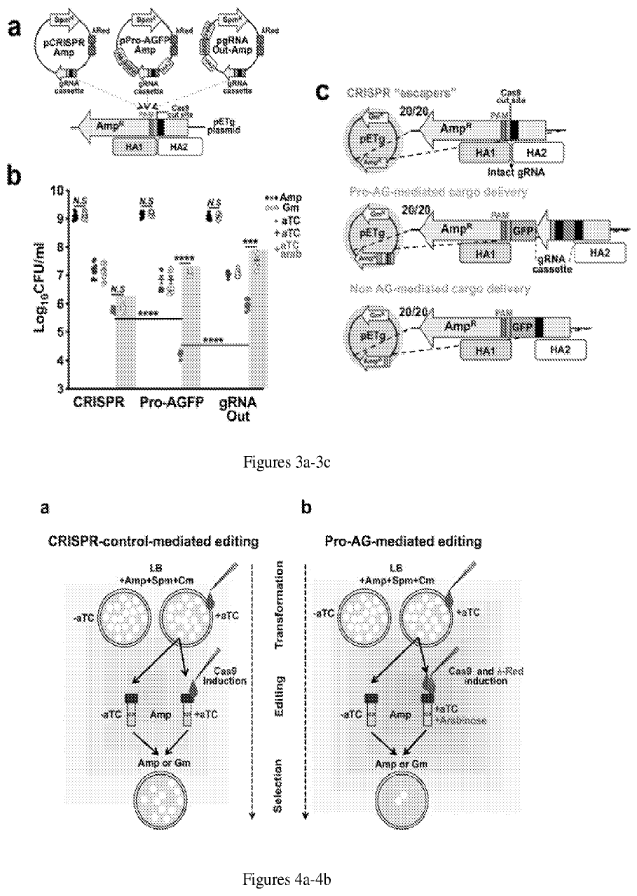

Initial transformation of E. coli with the compatible three-plasmid system (scheme in FIG. 4a) did not lead to significant differences in CFU recovery on triple antibiotic (Amp+Spm+Cm) agar plates either in the presence (+aTc) or absence (−aTc) of anhydrotetracycline induction with either of two different gRNAs targeting bla sequences (FIG. 5a). Individual E. coli colonies carrying all three plasmids were then tested under an overnight growth protocol that imposes multi-generational antibiotic selection (see FIGS. 4a-4b and Methods). Briefly, colonies inoculated in Luria broth (LB) were grown overnight, maintaining antibiotic selection with or without aTc induction (FIG. 4a). While terminal culture densities were unaltered by Cas9 induction (FIG. 5b), culture aliquots regrown under the more stringent conditions presented by selection on triple-antibiotic plates displayed significant Cas9-dependent reductions in CFU for both gRNA1 (˜10-fold) and gRNA2 (˜100-fold) (FIG. 1b). Differential responses to the Cas9 induction regimen are likely attributable to non-autonomous antibiotic rescue of mixed cells by secreted Bla in overnight liquid culture, absent during antibiotic plate selection of well isolated colonies. This strategy using CRISPR-Cas9 alone (e.g., without a homology template) is referred to hereafter as CRISPR-control.

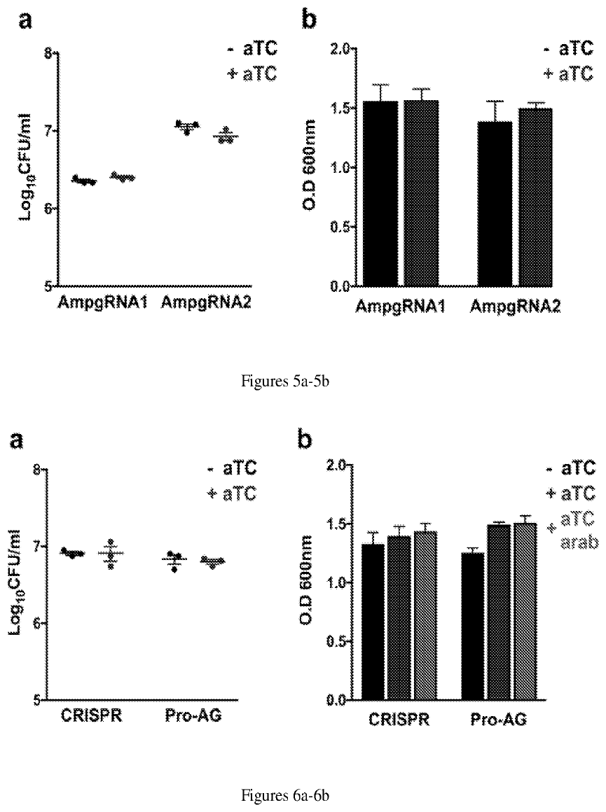

As the targeting efficiency of gRNA2 under the aforementioned CRISPR-control framework matched previously reported reductions in CFU using a similar experimental paradigm¹⁰, this gRNA was chosen to test in a “pro-active” configuration (Pro-AG) (FIG. 1c). The Spm^{R} gRNA-expressing plasmid was modified by flanking the gRNA2 expression cassette with bla sequence homology arms (HA, ˜500 bp each, comparable in length to HA employed in eukaryotic systems) that directly abutted the gRNA cleavage site, generating the Pro-AG plasmid (Table 1). Since the gRNA cleavage site is absent from this plasmid, the encoded gRNA is unable to cleave these sequences in the presence of Cas9. Because standard E. coli strains do not support efficient homology-based insertion of genomic cassettes following induction of double strand DNA breaks^{13,14}, a cassette encoding recombinogenic lambda-Red (λRed)^{15,16} enzymes under control of an arabinose inducible promoter (arab) that is also encoded on this plasmid⁹ was used. Plasmids pET (carrying the bla Amp^{R} target gene) and pCas9 were identical to those used for the “CRISPR-control” regimen (FIG. 1c). Following the parallel experimental scheme depicted in FIG. 4b, no significant differences in CFU following initial transformation of the three Pro-AG plasmid components under triple-antibiotic (Amp+Spm+Cm) selection with or without Cas9 induction (i.e., ±aTc) (FIG. 6a) was observed. Similarly, Cas9±λRed induction with aTc or arabinose, respectively, did not appreciably impact overnight culture densities under triple antibiotic selection (FIG. 6b).

### Pro-AG Efficiently Inactivates Antibiotic Resistance

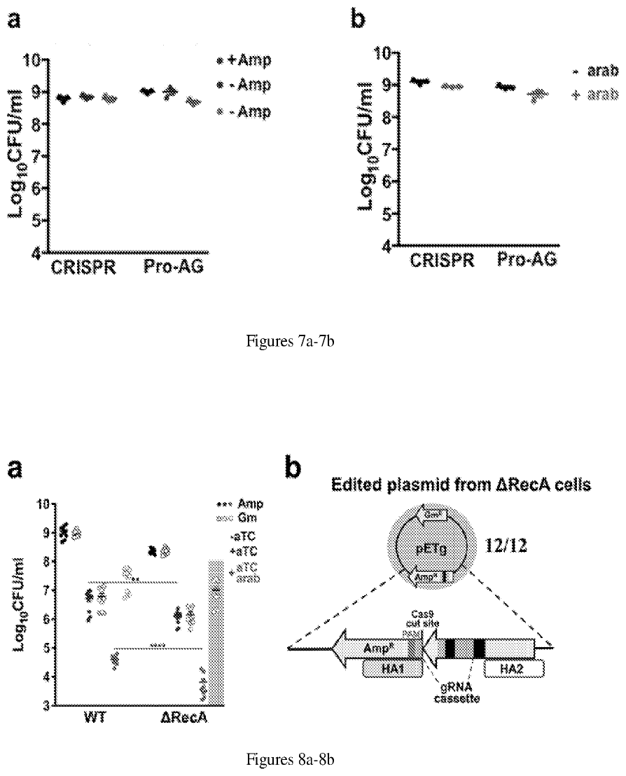

As an initial comparison between the CRISPR-control (FIG. 1a) and the Pro-AG (FIG. 1c) configurations, overnight cultures of E. coli colonies transformed with the corresponding three-plasmid systems were grown in triple antibiotics with or without aTc (to induce Cas9), alone or in combination with arabinose (to induce λRed enzymes), then plated for CFU enumeration (FIG. 1d). In contrast to ˜100-fold CFU reduction observed with the gRNA using the “CRISPR-control” component configuration (FIGS. 1b, d), targeting of bla using the Pro-AG format (i.e., induction of Cas9+λRed) led to a ˜100,000-fold reduction in Amp^{R} CFU (FIG. 1d). For reasons that remain unclear, induction of λRed also yielded a modest reduction in CFU recovery in the CRISPR-control regimen; however, this effect was much more pronounced in a Pro-AG context (FIG. 1d, green vs. blue greyscales shading). The intensified reduction in Amp^{R} E. coli CFU could not be attributed to secondary effects of aTc and/or Cas9 induction, since no significant differences in CFU were observed under Amp selection (+Amp) without Cas9 induction (−aTc) compared to Cas9 induction (+aTc) without Amp selection (−Amp) (FIG. 7a). Additional controls excluded suppressive effects of arabinose or λRed enzyme expression per se, as arabinose induction alone did not alter CFU recovery (FIG. 7b).

It was expected that either unrepaired CRISPR-mediated double-stranded DNA breaks or potential Pro-AG-mediated insertion of gRNA2 sequences into the corresponding cleavage site would inactivate the bla target gene, and therefore that E. coli undergoing such events would not be recovered upon selection for Amp^{R}. Indeed, among the few Amp^{R} colonies recovered following either CRISPR-control (green greyscale box) or Pro-AG (blue greenscale box) treatments (i.e., induction of Cas9+λRed), sequence analysis showed that 100% (30/30) carried unaltered pET plasmids with intact bla gene coding sequences at the gRNA target site (FIG. 1d, bottom panel). These rare examples of Amp^{R} E. coli colonies that evaded CRISPR or Pro-AG editing, likely from incomplete target cleavage of the high copy number plasmid, are referred to hereafter as “escapers”.

### Pro-AG Efficiently Reduced AR Incidence

A three-component split system was similarly devised (FIGS. 16a-16c) comprising: 1) a conditional Cas9-expressing regulon (inducible by anhydrotetracycline −aTC) carried on low copy-number plasmid-A; 2) a gRNA cassette (±cargo, denoted as <gRNA>) with (Pro-AG) or without (CRISPR control) flanking homology arms (HA), comprising sequences adjacent to the cut site of the beta-lactamase (bla) AR gene, carried on low copy-number plasmid-B,C; and 3) an arabinose-inducible λRed DNA repair cassette, also encoded by plasmid-B,C³³. As a target for Pro-AG, the high copy number plasmid-pET selectable by AR genes conferring resistance to ampicillin (Amp^{R}=bla target) and gentamicin (Gm^{R}) was used (FIG. 16a).

This Pro-AG system produced a ˜10⁵-fold reduction in Amp^{R} CFU in initial experiments, which represents a ˜100-fold improved efficiency over CRISPR-only controls. A series of controls ruled out potential confounding generalized effects resulting from Cas9 and/or λRed expression. Three key experiments indicating that the Pro-AG system acted through a self-amplifying process were then performed, which depended in part on increasing the gRNA cassette copy number by its precise insertion into the bla target gene on the high-copy number pET plasmid³³. Experiment 1: exploiting the dual selection (Amp^{R} and Gm^{R}) of the pET target plasmid, colonies on Gm plates were recovered that did not grow on Amp (FIGS. 16b-16c), and all clones analyzed carried precise insertions of the gRNA cassette into the bla gene at the gRNA-directed cleavage site. Adding a GFP cargo gene along with the gRNA cassette in the Pro-AG plasmid-BC did not hinder its efficiency in reducing Amp^{R} CFU. Experiment 2: The highly efficient full Pro-AG configuration, where both gRNA and GFP lie between homology arms, “gRNA-In” (FIG. 17b) was compared to one in which the gRNA was placed outside of the homology arms (leaving only GFP inside) “gRNA-out” (FIG. 17c). The gRNA-Out configuration lost efficiency (FIG. 17d), performing no better than control-CRISPR in reducing Amp^{R} CFU (green vs. red data in FIG. 17d, grayscale), although all gRNA-out colonies recovered on Gm selection plates carried perfectly edited insertions of the GFP-only cassette into the bla target site. While the gRNA-Out configuration relied on the same precise λRed-dependent editing mechanism as the full Pro-AG (gRNA-In) system, it did not benefit from the gRNA amplification cycle associated with the gRNA-In configuration, and hence did not outperform control CRISPR in reducing Amp^{R} CFU. In Experiment 3, the temperature sensitivity of plasmid-BC replication was exploited³³. Pro-AG and control CRISPR experiments were initiated briefly at the permissive temperature to allow for transfer of the gRNA from plasmid-BC to the target plasmid pET, and then shifted to the non-permissive temperature. Under these conditions, only Pro-AG retained significant capacity to reduce Amp^{R} CFU.

The above experiments rigorously validate that Pro-AG acts by copying the gRNA±cargo cassette precisely into the bla gene target site, and does so via a positive feedback loop driven in great part by self-amplification of the gRNA transgene. Further experiments revealed that Pro-AG also efficiently edited a single-copy genomic target locus (lacZ) by precise insertion of a gRNA±cargo cassette³³. There was no difference in editing efficiency between the full Pro-AG and gRNA-out configurations, consistent with the expectation that amplification of the gRNA should not be required for editing single-copy targets.

The fact that Pro-AG elements carry CRISPR-Cas9 components optimally configured to copy themselves as selfish genetic elements into both high-copy number and single copy-number target genes is highly innovative. Pro-AG is a breakthrough technology enabling highly efficient and precise gene editing that out-performs standard cut-and-destroy CRISPR approaches by more than 100-fold to reduce Amp^{R} carried by the high-copy number pET plasmid. The Pro-AG system brings forward many potential novel applications including: next generation anti-AR tools for efficient editing of plasmid and genomic loci to scrub AR from human bacterial pathogens or from the environment (e.g., feed lots, fish farms, water treatment plants); re-establishing healthy microbiomes; and developing synthetic logic-gated bio-circuitry and amplifier circuits.

### Pro-AG Results in Precise Homology-Mediated Editing

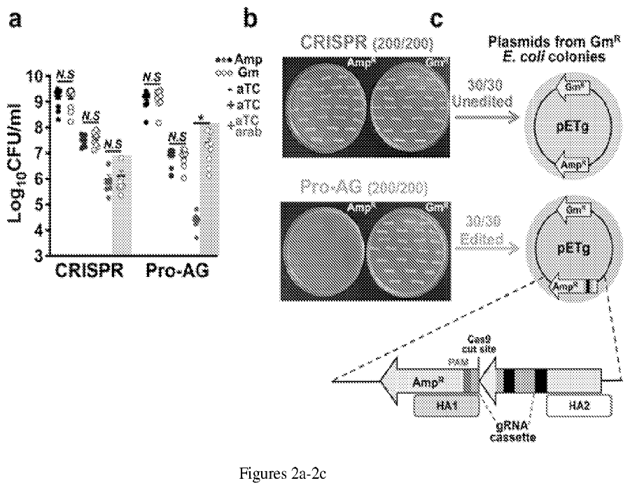

In analogy to HDR-dependent copying of gene-drive elements in diploid organisms, enhanced gene targeting activity of Pro-AG over CRISPR-control (FIG. 1d) might reflect homology-mediated insertion of the gRNA cassette into the bla gRNA2/Cas9 cleavage site. This hypothesis was tested by incorporating a second selectable antibiotic resistance gene (gentamicin-Gm^{R}) into the target pET plasmid (pETg; Table 1), allowing recovery of bla gene-edited plasmids by selection on Gm plates (FIGS. 4a-4b). Consistent with high efficiency insertional copying, virtually the entire decrement of CFU seen in Pro-AG vs. CRISPR-control systems under Cas9+λRed induction and Amp^{R} selection could be recovered under Gm^{R} selection (FIG. 2a, blue greyscale box). Likewise, as expected, all Pro-AG recovered Gm^{R} colonies failed to grow on Amp plates (FIG. 2b, bottom panel). In contrast to the nearly full restoration of the λRed-induced CFU decrement observed upon Gm^{R} selection with the Pro-AG regimen, no CFU recovery was noted under Gm^{R} vs. Amp^{R} selection using the CRISPR-control protocol for targeting bla (FIG. 2a, green greyscale box). A clear prediction of the Pro-AG hypothesis examined above is that all recovered Gm^{R} colonies should carry a precise insertion of the gRNA expression cassette. Indeed, analysis of 30 Gm-selected pETg plasmids from Pro-AG single colonies confirmed that the DNA cassette carried between the two homology arms, including the full gRNA2 scaffold and its promoter (FIG. 2c, bottom scheme), were perfectly copied from the donor plasmid (pPro-AGAmp, FIG. 1c; Table 1) into the gRNA2 cleavage site of the bla target gene in all clones (FIG. 2c, blue greyscale circle). Thus the function of the targeted bla gene is disrupted by insertion of the gRNA expression cassette within its protein coding region. In contrast, all CRISPR-control treated unedited E. coli escaper colonies recovered on Gm^{R} selection regrew under selection for Amp^{R} (FIG. 2b, top panel). Consistent with such escapers having evaded CRISPR-mediated mutagenesis, all (30/30) examined Amp^{R}+Gm^{R} CRISPR escapers displayed intact (unedited) bla gRNA2 target sites (FIG. 2c, green greyscale circle). In aggregate, the above findings support the hypothesis that the greatly enhanced reduction of Amp^{R} CFU observed upon Pro-AG versus CRISPR-control editing configurations is quantitatively attributable to homology-mediated insertion of gRNA2 sequences into bla coding sequences on the target pETg plasmid. Of note, each insertional editing event expands the pool of functional gRNA donor scaffolds with extended homology arms in their newly copied plasmid context, which may initiate a chain reaction accelerating further insertional events.

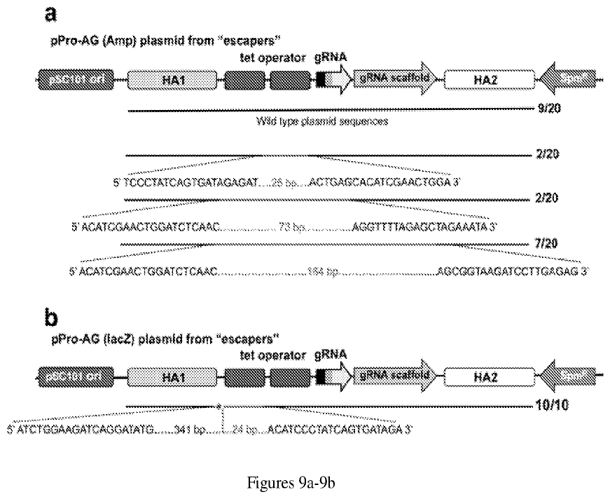

As mentioned above, E. coli “escaper” colonies that evaded Pro-AG editing showed intact target sequences (FIG. 1d, bottom panel). It was hypothesized that inefficient Cas9 cleavage in escaper cells could reflect competition with the bacterial DNA repair system. This idea was tested by comparing Pro-AG editing of the bla gene target carried by the pETg plasmid in WT vs. ΔrecA E. coli (FIG. 4b). A significant reduction of Amp^{R} CFUs was obtained in ΔrecA cells, under Cas9 induction alone or in combination with λRed (FIG. 8a). All target plasmids analyzed from both Gm^{R} WT and ΔrecA E. coli colonies bore precise gRNA2 insertions into bla target gene (FIG. 8b), paralleling the editing efficiencies observed in previous experiments (FIG. 2c). While these findings indicate that RecA is not required for copying the gRNA2 expression cassette from donor plasmid to target gene, they also suggest that Pro-AG escaper cells are partially protected in a RecA-dependent manner. An additional previously observed mechanism of escape from CRISPR-Cas9-mediated editing of E. coli involves mutations in the gRNA-harboring plasmid^{8,10}. Consistent with similar mechanisms operating in CRISPR-based editing experiments, analysis of the pPro-AG (Amp) plasmid from escaper cells revealed that ˜50% of gRNA launching plasmids recovered from escaper lines harbored deletions spanning the operator region, the gRNA and/or the gRNA scaffold sequences (FIG. 9a).

Pro-AG Depends on Homology Sequences Flanking the gRNA

The above described Pro-AG configuration, wherein the gRNA expression cassette is flanked directly by homology arms, represents the minimal possible self-copying element. This system might also be exploited to deliver additional DNA cargo sequences efficiently. As a test case, a green fluorescence protein (GFP) transgene was included as cargo with the gRNA2 expression cassette between the bla gene homology arms (pPro-AGFPAmp, FIG. 3a, Table 1). Using the same experimental design described above for targeting Amp^{R}+Gm^{R} plasmid pETg with the “gRNA-only” pPro-AG plasmid (FIG. 3A), the cargo-laden pPro-AGFP construct performed similarly to its minimal counterpart in reducing Amp^{R} CFU versus the CRISPR-control regimen (FIG. 3b). As for the minimal Pro-AG element, regrowth of single Pro-AGFP colonies recovered on Gm plates following Cas9+λRed induction (FIG. 3b, blue greyscale box) revealed perfect insertion of the composite gRNA2:GFP cassette into the bla gRNA2 cleavage site in 100% of clones analyzed (FIG. 3c, blue greyscale circle). Again, escaper colonies recovered under the CRISPR-control regimen on Gm plates with Cas9+λRed induction (FIG. 3b, green greyscale box) maintained pristine unedited gRNA target sequences and also displayed an Amp^{R} phenotype (FIG. 3c, green greyscale circle). Thus, the Pro-AG system leads to highly efficient and precise insertion of the gRNA2:GFP cargo bearing cassette into the gRNA2 bla gene target site.

A defining feature of self-amplifying CRISPR-based gene-drive systems in diploid organisms is insertion of the gRNA-bearing cassette at the exact genomic site where the gRNA directs target cleavage¹⁷. If amplification of the gRNA gene dosage resulting from its being actively copied contributed to the enhanced efficiency of the Pro-AG system, then placement of the gRNA outside of the homology arm cassette (pgRNA-Out-Amp) (FIG. 3a, Table 1) would significantly reduce editing efficiency of the target plasmid. Indeed, the gRNA-Out configuration performed comparably to the CRIPSR-control protocol, leading only to an approximately 3 log₁₀-fold reduction in CFU under Amp^{R} selection and Cas9+λRed+induction (FIG. 3b). Thus, the entire boost in Amp^{R} CFU reduction provided by the Pro-AG system was abrogated by placing the gRNA outside of the homology arms, consistent with the hypothesis that the Pro-AG process acts via a positive feedback amplification mechanism (FIG. 3b). Although the gRNA-out configuration eliminated the ˜2-log increment in CFU reduction observed with Pro-AGFP arrangement under Amp^{R} selection, significant CFU recovery was nonetheless observed upon Gm^{R} selection, suggesting that a large fraction of the baseline DNA breaks induced by CRISPR-control conditions were now being repaired by precise copying of the homology flanked GFP-only cassette. Indeed, all 20 plasmids analyzed from Gm^{R} colonies under the gRNA-Out regimen (FIG. 3b, red greyscale bar) carried perfect insertions of the GFP-only cassette within the gRNA2 cleavage site of bla (FIG. 3c, red greyscale circle).

Amplification of the gRNA Contributes to Pro-AG Performance

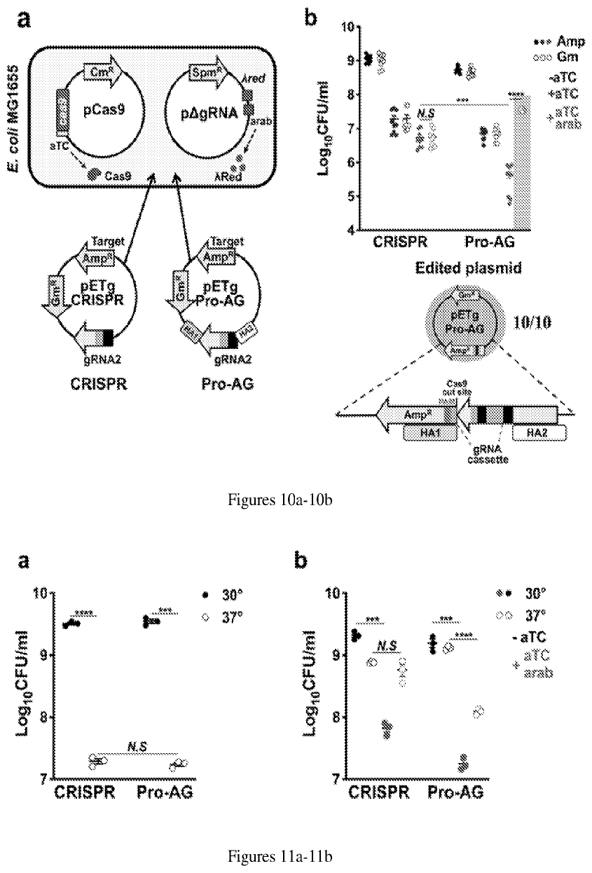

As copying of the gRNA2 expression cassette from a low (pPro-AG) to a high copy number plasmid (pETg) significantly amplified gRNA gene number and hence expression levels, it was asked whether the enhanced performance of Pro-AG versus CRISPR-control could be wholly attributable to this effect. This question was addressed by comparing Amp^{R} CFU reduction using CRISPR-control versus Pro-AG configurations in a situation where the bla-targeting gRNA2 was encoded from the outset on a high-copy number plasmid, namely the pETg target plasmid itself, which would limit gRNA amplification to at most 2-fold. E. coli harboring pCas9 and pAgRNA (Table 1) expressing Cas9 and λRed, respectively, were transformed with either pETgCRISPR or pETgPro-AG plasmids (FIG. 10a, Table 1). Using earlier CRISPR-control and Pro-AG editing protocols under Amp selection (FIGS. 4a-4b), Pro-AG performed 1-logfold more efficiently than CRISPR-control in reducing Amp^{R} CFUs (FIG. 10b). While this Pro-AG gain is less than that achieved when gRNA2 was launched from a low copy plasmid, where a 2-logfold improved efficiencies were observed, this more modest enhancement suggests that the full Pro-AG effect is not solely attributable to copy number amplification of gRNA2 (FIG. 1D). As before, significant CFU recovery occurred on Gm plates following Pro-AG editing (FIG. 10b, top panel, blue shading, grayscale), with perfect insertions of gRNA cassette into bla coding sequences in 100% of pETgPro-AG plasmids analyzed (FIG. 10b, bottom panel). Therefore, amplification of the homology-flanked gRNA, which may replicate itself ˜50 times in a high-copy plasmid, contributes significantly to the enhanced performance of Pro-AG. Additional mechanisms such as increased homology sequence length and double strand DNA break repair may also contribute to enhanced Pro-AG efficiency compared to CRISPR only controls.

### Pro-AG Acts Via a Self-Amplifying Mechanism

As yet another approach to assess the role of cassette amplification in the enhanced performance of Pro-AG versus CRISPR-control paradigms the temperature sensitive nature of replication of the low-copy number gRNA2 donor plasmids was used. Switching overnight growth cultures from 30° C. (permissive temperature for plasmid replication) to 37° C. (non-permissive temperature) during the editing protocol reduced Spm^{R} CFU that harbor the gRNA2 plasmid by ˜2.5-logfold (FIG. 11a). As in previous experiments, Amp selection produced a significant reduction of Amp^{R} CFUs for both CRISPR-control and Pro-AG configurations following Cas9 and λRed induction (+aTc +arab) at 30° C., which was more pronounced for Pro-AG. In contrast, only the Pro-AG regimen, and not CRISPR-controls, also reduced Amp^{R} CFUs following Cas9+λRed induction (+aTc +arab) at 37° C. (FIG. 11b). Based on these several independent lines of corroborating evidence, it is concluded that incorporating the gRNA (±cargo) between homology arms to generate the active genetic cassette greatly increases targeting efficiency of a high-copy number plasmid via a self-sustaining positive feedback loop.

### Pro-AG is Amenable to Manipulation of Large Plasmids

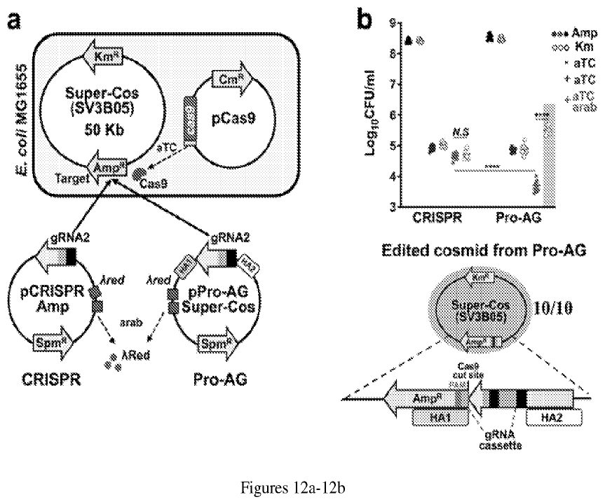

Large plasmids carried by various pathogens pose an important health problem and are challenging to manipulate by traditional methods. An E. coli strain harboring the pCas9 plasmid and a ˜50 Kb cosmid vector (Supercos SV305, Table 1) carrying Amp and Km selection markers were chosen as a test case. As in previous experiments, cells were transformed with either the pCRISPR-Amp control or pPro-AG (SuperCos) plasmids, the latter adopting a Pro-AG configuration to target the bla gene encoded on the cosmid (FIG. 12a). Following the standard editing protocols (FIGS. 4a-4b), a 1-logfold greater reduction in Amp^{R} CFUs was observed with the Pro-AG regimen than CRISPR-control following Cas9 and λRed induction (+aTc +arab) (FIG. 12b, top panel). Indicative of Pro-AG-mediated double strand DNA break repair, significant CFUs were rescued under Km selection (FIG. 12b, top panel, blue shading box, grayscale), and 100% of the Supercos SV305 cosmids isolated and sequenced showed a precise gRNA cassette insertion into bla (FIG. 12b, bottom panel). Pro-AG is thus well suited for gene editing large multi-copy plasmids, with efficient homology-mediated insertion of gRNA sequences into the targeted coding region.

### CRISPR and Pro-AG are Equivalent for Single Locus Editing

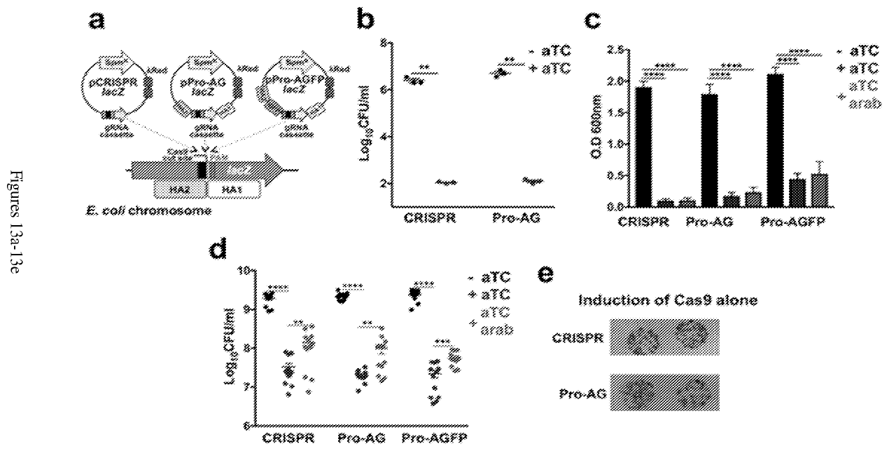

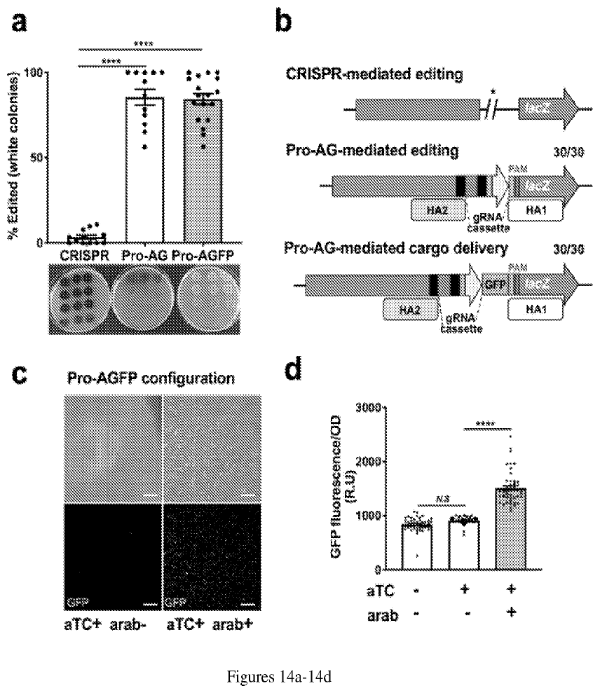

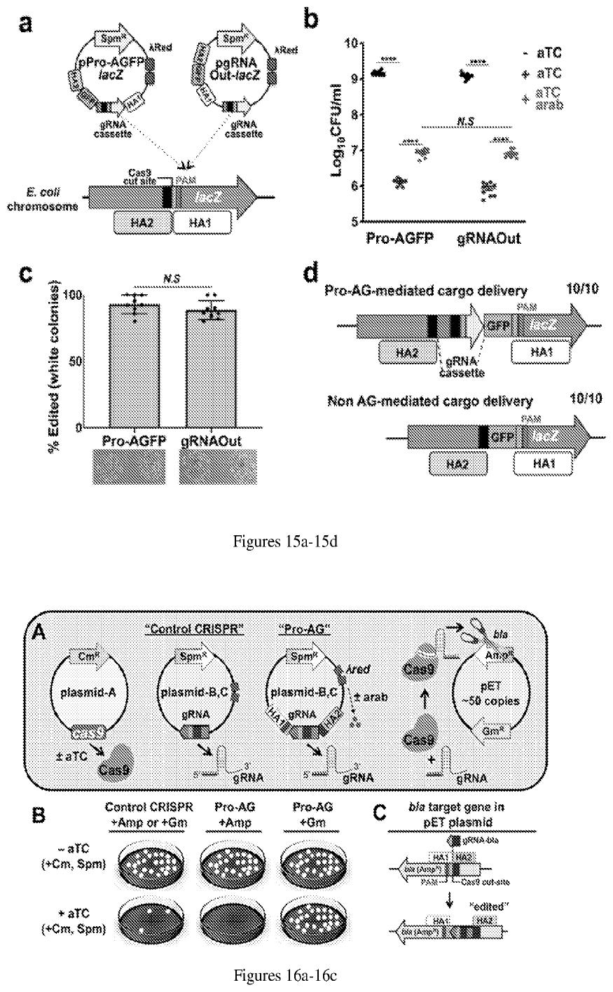

Given its efficiency in meeting the challenging task of targeting a high and moderate copy number plasmids, whether the Pro-AG approach could similarly be employed to insert gRNA-only or gRNA+GFP cargo cassettes into a single copy chromosomal target (e.g., the lacZ gene) was asked. It was found that Pro-AG configurations indeed performed with high efficiency in this context as well (FIGS. 13a-13e and 14a-14d). Since in this context, there is no need for cassette amplification provided by Pro-AG, it was predicted that targeting efficiency would not depend on the placement of the gRNA between homology arms. This expectation was confirmed by experiments in which the gRNA was placed between (gRNA-in) or outside (gRNA-out) of the GFP-containing cassette since both configuratiperformed equivalently in precise editing of the lacZ target gene (FIGS. 15a-15d).

Cumulatively, these results indicate that the Pro-AG configuration is 2-3 orders of magnitude more efficient in disrupting the activity of a high copy-number target gene (bla) than the CRISPR-control arrangement, yielding to a 4-5 log₁₀-fold reduction in Amp^{R} E. coli, fully attributed to precise insertion of the gRNA (±cargo) cassette from the editing vector into the targeted gRNA cleavage site. Increased potency of the Pro-AG versus CRISPR-control configuration in reducing antibiotic resistant CFUs depends on the gRNA cassette being precisely flanked by bla homology arms, suggesting that an amplifying positive feedback loop occurs from increasing gRNA copy number, a potential rate limiting component of the system. The high efficiencies of accurate Pro-AG cassette copying in bacteria are comparable to those attained by gene-drive systems in diploid organisms (>90%)^{1-3,18}. In addition to its high efficiency, Pro-AG maintains an ability to perform precise and potentially subtle edits of a target gene rather than simply eliminating the target sequence or bacteria carrying that locus.

Multiple genome engineering applications could ultimately benefit from incorporation of the Pro-AG platform, including elimination of bacterial virulence factors carried by diverse episomal elements and resistance determinants in commensal bacteria as well as different prokaryotic pathogens, scrubbing antibiotic resistance genes from bacteria in the environment¹⁹, livestock, inland fish farms, or sewage treatment ponds²⁰, reprogramming genetic circuits impacting bacterial physiology, or tailoring interactions among the microbiota in environmental or host niches. For each of these applications suitable delivery systems such as phage vectors²¹ or conjugative plasmids²² would need to be developed and tailored to the specific contexts in which they were being deployed.

### The Development of Multi-Targeting Pro-AG Strategies

Pro-AG editing may be extended to more than a single target, enabling the design of integrated editors that target multiple components contributing to AR/virulence. As a first step in this effort, a dual Pro-AG systems may edit two plasmid targets or plasmid+genomic targets. Such dual targeting systems could either inactivate two non-essential loci (e.g. AR and virulence factors), or inactivate one locus and alter the activity of another (e.g., a gene encoding an essential transporter).

AR phenotypes typically result from complex combinations of several genetic alterations including a bona fide AR gene causing degradation or inactivation of the antibiotic, and accessory genes encoding altered membrane transporters, or efflux pumps, which more effectively exclude or eject antibiotics, respectively. In addition, bacterial pathogens often express virulence factors including toxins that alter host responses to favor pathogen persistence or host invasion.

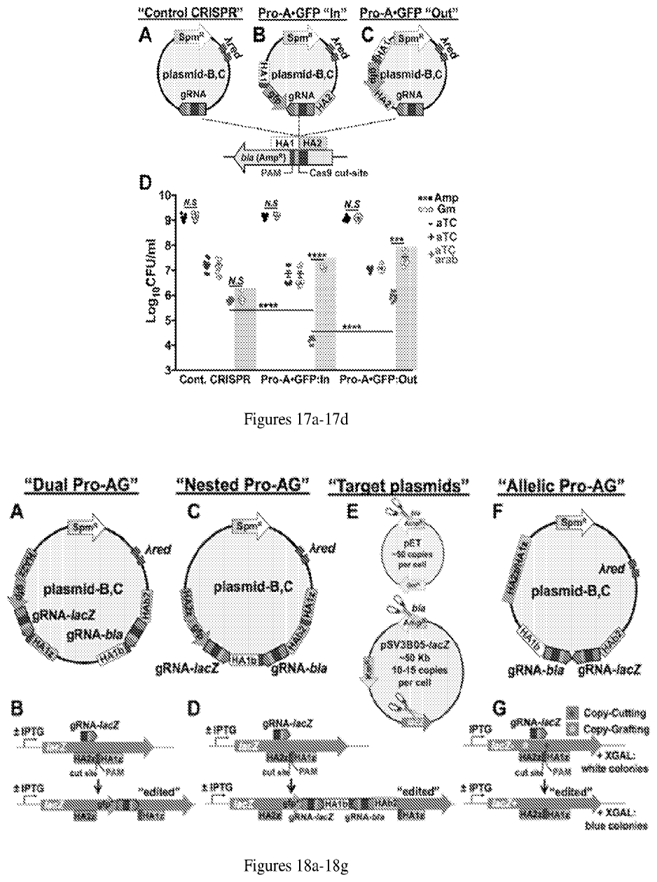

Dual editing Pro-AG systems may be constructed that carry two separate gRNA cassettes designed to inactivate different non-essential target genes. Each of these two cassettes will be flanked by homology arms derived from their corresponding targets (FIG. 18a). One cassette will carry the standard gRNA (gRNA-bla) and the second cassette will target either a chromosomal non-essential locus (e.g., lacZ or an established virulence factor such as type 1 fimbrial adhesis, fimH) or an accessory plasmid-encoded factor such as an efflux pump carried on a low-copy number plasmid (e.g., the 50 kb Super-Cos plasmid SV3B05). The plasmids shown in FIG. 18e may be used. An advantage of this experimental design is that AR resistance and GFP expression may be tracked (FIG. 18b) as independent editing outcomes since only precise lacZ editing generates an in-frame βGal-GFP fusion protein³³. These experiments permit quantification of the relative editing efficiencies of the bla and lacZ genes in the contexts of differing target copy-number (e.g., high-copy number pET bla target plus single copy genomic lacZ target, and both bla and lacZ targets carried on the mid-copy number pSV3B05 plasmid). Next, inactivation of the high copy number bla gene carried on the pET plasmid and the single-copy genomically encoded FimH pilus-protein virulence factor from uropathogenic E. coli (UPEC-UTT89) may be evaluated. Mutations in fimH or its pharmacological targeting abolish the ability of UPEC to colonize the bladder and to form intracellular bacterial colonies and quiescent intracellular reservoirs.

An alternative to designing two separate HA-flanked gRNA cassettes is the “nested” configuration, in which both gRNAs are carried on a single compound cassette (FIG. 18c). In this arrangement, an inner HA-b flanked cassette (carrying gRNA-bla) is contained as a unit within an outer HA-z flanked cassette (also carrying gRNA-lacZ and gfp). Induction of Pro-AG by aTC and arabinose should lead to the full outer cassette (FIG. 18d) being inserted into the lacZ locus. This edited lacZ locus could then serve secondarily as a genomic source from which to launch the inner anti-Amp^{R} bla-targeting cassette. The efficiencies of editing both the bla and lacZ targets may be compared using the dual versus nested Pro-AG configurations. The systems may be challenged with different copy-number targets (i.e., pET bla+genomic lacZ versus bla+lacZ on the pSV3B05 plasmid) and assessed launching <gRNA> cassettes from genomic sites (e.g., inserted into the lacZ locus). An advantage of retaining a genomic copy of the Pro-AG cassette (which could also be engineered to include Cas9) is that it should provide persistent immunity to future acquisition of plasmids carrying AR factors, as do endogenous CRISPR systems.

In addition to insertional gene inactivation, one may wish to edit specific nucleotides. For example in the case of fimH, point mutations have been identified that increase epithelial adherence, resulting in enhanced virulence. Thus, reinstating the wild-type allele of fimH could contribute to effective control of E. coli infections such as UPEC. For such subtle edits, “allelic Pro-AG” strategies may be tested (FIGS. 18g and 18f) analogous to those developed in Drosophila³⁴ using lacZ again as a proof-of-concept. For these experiments two gRNAs, <gRNA-bla, gRNA-lacZ>, are placed between the bla HA. Two alternative forms of allelic drive may be tested that referred to as “copy-cutting” and “copy-grafting”. For copy-cutting, a gRNA may be designed that specifically cuts a mutant lacZ allele (e.g., a stop codon creating a new PAM site absent in the wild-type sequence: burgundy (grayscale) asterisk in FIG. 18g). The gRNA-lacZ selectively targets the mutant allele (either present in the genome or on pSV3B05 plasmid), but not the refractory wild-type donor fragment carried on plasmid-BC (FIG. 18f), leading to cleavage, editing, and restoration of lacZ function. For copy-grafting, the mutant allele is located outside of the gRNA target site a short distance away (<25 bp, denoted by yellow asterisk in FIG. 18g). Here, a synonymous cut-resistant mutation is made in close proximity to the wild-type lacZ donor template, protecting it from cleavage and allowing it to serve as a template to repair the targeted mutant allele. Because of its minimal sequence constraints, copy-grafting can be implemented more generally than copy-cutting. It is expected that these allelic drive strategies will be efficient in bacteria given the parallel activities of gene-drive systems in eukaryotes and Pro-AG in E. coli.

### Pro-AG Dependent Genetic Relay and Switch Circuits

The salient novelty of Pro-AG is its ability to copy a gene cassette with great fidelity from one DNA molecule to another. This intrinsic property of Pro-AG has several impactful applications for designing synthetic genetic circuits. Two types of integrated Pro-AG circuit elements may be built: 1) relay systems based on sequential copying events resolved by a conditional output from the last step, and 2) threshold gated amplifier circuits. These new temporally sequenced and variable-gain circuit elements should be of broad utility in designing next-generation synthetic biology circuits including ordered logic circuits and exquisitely discriminating biosensors for future biomedical applications.

Because the homology arms flanking gRNA cassettes can be chosen to cover only a subset of a functional gene sequence, one can design an element (FIG. 19a) with minimal homology arms (HA1 and HA2) carrying two gRNAs, the first of which (gRNA1) copies the element to a target carrying the entire gene sequence (FIG. 19b). Insertion of the cassette into this primary target (FIG. 19c) then endows that edited locus with the capacity to serve as a “launching pad” using outside homology arms (HA3 and HA4) for copying the cassette into second target using gRNA2 (FIGS. 19d-19e). The copied cassette also carries a transgene encoding GFP, but with a minus-1 frameshift so that once inserted into the plasmid bla locus (FIG. 19c) it will not produce a functional fluorescent fusion-protein. The second bla target (cut by gRNA2), however, carries an opposite plus-1 frameshift. If the cassette copies as planned from the plasmid into the truncated and frame-shifted genomic bla target, a functional GFP fusion product should be produced. Thus, GFP activity is observed if, and only if, a successful sequential relay copying event occurred.

Constitutive expression of both gRNA1 and gRNA2 (e.g., by the Tet promoter) could result in unedited gRNA2-mediated genomic cleavage events leading to cell death and reducing the total number of recovered CFU. One variable that may be manipulated to avoid this potential problem and optimize the relay process is to place gRNA2 under the control of a second conditional promoter (e.g., the IPTG inducible lac promoter) whose expression by be activated once the cassette has been copied into its first pET plasmid target. Control experiments include: 1) omitting the intermediate pET target plasmid (which should result in high cell lethality upon Cas9 induction due to gRNA2 cleaving the genomic target without an available repair template), and 2) omitting gRNA2, which should result in a viable outcome, but not in copying of gfp into the genome (i.e., all colonies should remain GFP negative).

Pro-AG amplified gated switches. Amplifier circuits are ubiquitous in electronics, comprising over half of the block units in analog radios and televisions. As summarized above, the Pro-AG system is a genetic counterpart of an amplifier since it increases its frequency (e.g., amplitude) and inactivates AR (the waveform) without altering other properties of the signal (e.g., as illustrated by the gRNA-in versus gRNA-out experiment). A simple biosensor to detect the presence of plasmids in bacterial cells using an amplifier circuit constructed from Pro-AG and toxin-antitoxin system components may be built. Toxin-anti-toxin (TA) systems employ exquisitely balanced transcriptional regulatory networks to control the synthesis of counteracting protein products. For example, type-II protein TA systems employ short lived anti-toxin and stable toxin pairs that function as “addiction systems”, which if lost, result in bacterial cell intoxication and death. Typically, in type II TA systems, the anti-toxin acts as a low-affinity repressor of the operon encoding both anti-toxin and toxin, but becomes a high-affinity repressor when bound to the toxin. In the well characterized MazEF system in E. coli, binding of MazE anti-toxin to the MazF toxin (an endoribonuclease targeting specific cellular mRNAs to trigger cell death) acts as a potent repressor when bound to a 12 nucleotide operator-a motif within the 77 bp MazEF promoter.

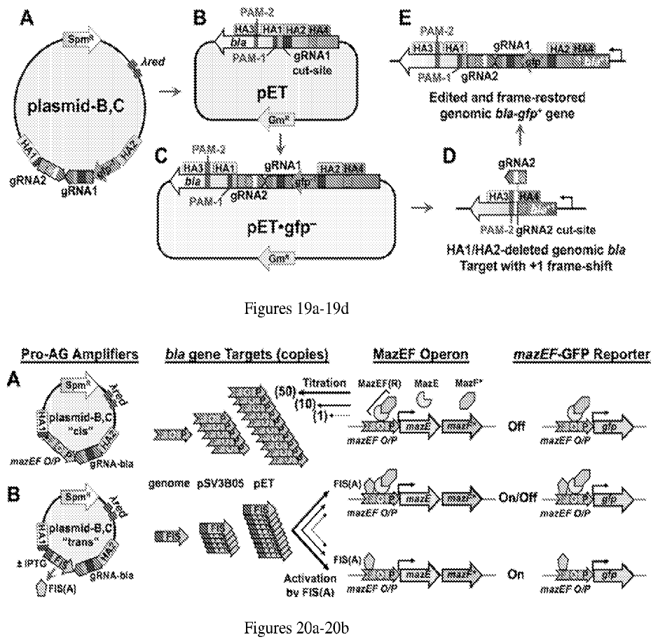

Two amplifier circuits (cis and trans) can control expression of reporter-GFP coding sequences fused to the MazEF operator/promoter carried with an intact MazEF TA system present at differing copy number (e.g., genomic: ˜1 copy; pSV3B05: 10-15 copies; and pET: ˜50 copies) (FIGS. 20a-20b). To prevent this system from killing bacteria, the enzymatic activity of the toxin component may be inactivated with a mazF*point mutation (e.g., E24A) that does not alter its binding to MazE. For the cis-circuit (FIG. 20a), either the full 77 bp MazEF promoter or just the 12-nucleotide operator MazEF binding site may be instered (as single or multimerized elements) as cargo on the HA flanked gRNA vector (plasmid-BC). As the gRNA cassette copies itself into high-copy number targets, the corresponding increase in the copy number of the MazEF binding sites should titrate out the MazEF repressor, resulting in de-repression of gfp-reporter gene expression and hence, in GFP+ bacterial cells.

For the trans-circuit (FIG. 20b), use of positive regulation of the MazEF operon by the transcription factor FIS, which binds to sequences at the 5′ end of the MazEF promoter may be used, which can regulate MazEF expression in a dose dependent fashion. FIS coding sequences may be inserted under control of the lac promoter into the HA-flanked gRNA cassette and to determine the level of IPTG required to induce GFP expression driven from the MazEF promoter. The level of IPTG may be reduced to a point where no GFP expression is detected, and using Pro-AG, copy the gRNA+FIS cassette into different copy number targets to assess the level of gene amplification required for induction of MazEF-gfp driven reporter expression.

### Incorporating Pro-AG on Plasmid Conjugal Delivery Systems

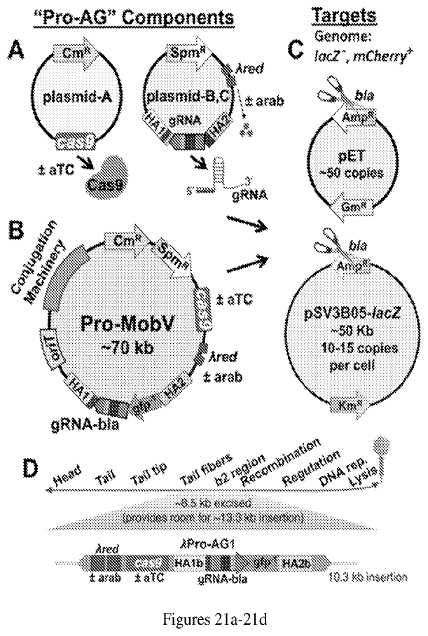

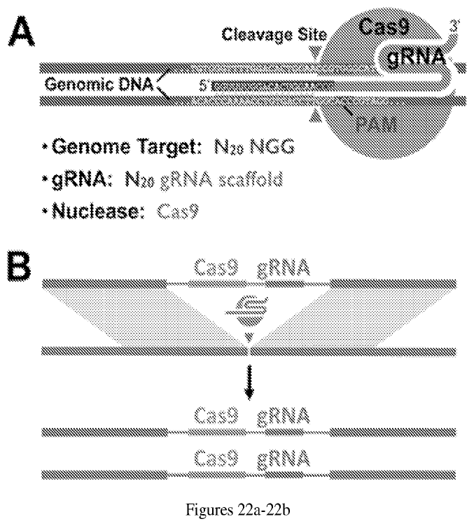

A wide array of conjugal systems reside on bacterial plasmids enabling their transfer and propagation, both in Gram- and Gram+ bacteria, and many more plasmids carry essential cis-acting elements (e.g., oriT recognized by TSS4 DNA binding components) enabling conjugation in-trans. A recent study²² demonstrated high conjugal transfer efficiencies of basic CRISPR machinery by a 61 kb cis-acting conjugal plasmid referred to as pNuc-cis. Combined Pro-AG components may be inserted into pNuc-cis to create the Pro-MobV conjugal plasmid (FIGS. 21a-21d).

Only the gRNA±non-functional gfp⁻¹ (a frameshift mutant expressing only an HA-tagged N-terminal fragment of the GFP protein) is flanked between homology arms to avoid toxicity due to over-expression of the full-length active GFP protein or the λRed cassette³³. The Pro-MobV system retains potent AR-reducing activity and the high efficiency of pNuc-cis in conjugal transfer to naïve AR bacterial recipients (in E. coli K12, uropathogenic E. coli-UPEC, Salmonella enterica, and S. typhimurium). A mCherry marker may also be inserted into the lacZ gene of recipient bacteria (using gRNA-lacZ to edit the endogenous lacZ gene) to permit identification of trans-conjugant cells³⁵. For these experiments, published protocols under planktonic (standard cultures) and biofilm promoting conditions (glass beads) are employed, the latter sustaining significantly enhanced conjugation²². Should the integrated Pro-AG-MobV plasmid not perform efficiently in reducing Amp^{R} CFU, varying the stoichiometry of core components by separating out either Pro-AG plasmid-A, carrying cas9, or plasmid-BC, carrying the HA flanked gRNA cassette and λRed cassette onto a low copy number plasmid, may improve performance of the system. If it does, a cis-acting oriT sequence should be added onto the plasmid carrying the isolated low copy number Pro-AG component and test co-transfer efficiencies of the cis and trans conjugal plasmids to recipient cells. The current Pro-AG components may be combined into Pro-MobV, and then updated with enhanced and/or dual effector systems to develop optimized Pro-AG conjugal systems suitable for in vivo testing.

With respect to the core Pro-AG components, it is expected that escapers of the Pro-MobV system may arise through mutations affecting either the Pro-AG/conjugal machinery itself or genomic loci essential for conjugal transfer. As systematic efforts to identify single locus mutations conferring resistance to conjugal transfer have failed³⁶, it is anticipated that the former class of mutations may dominate. The frequency of resistance mutations may be assessed by measuring the fraction of AR CFU following conjugal transfer of Pro-MobV to target cells carrying the lacZ-mCherry fusion marker. The source of resistance may be pinpointed in AR recipient cells (lacZ⁻, mCherry⁺): mutations in Pro-MobV plasmid components versus alterations in the bacterial genome. The genomes of refractory bacterial colonies are then sequenced to identify candidate mutated genomic targets.

Stable propagation of the Pro-MobV plasmid may be increased. For example, the additional cis-acting oriT sites mediating trans-conjugation may be incorporated by alternative pathways, should components of the TSS4 system be mutated or inhibited by some type of cytoplasmic process. Also, duplicating core Pro-AG components may reduce the incidence of conjugation competent but Pro-AG defective mutants.

Phage delivery offers a complement to conjugal transfer systems. Dissemination of the same Pro-AG components may be tested using lysogenic λ-prophage vectors to target high- and intermediate copy-number AR target plasmids in E. coli. An evolved temperate λ-phage may be used (λ^{LO}) with dual receptor binding (LamB and OmpF) that greatly reduces the incidence of envelop-derived forms of bacterial resistance to infection. Briefly, the standard Pro-AG components may be inserted: aTC-regulated cas9, arabinose-inducible λRed, and a bla-homology arm-flanked <gRNA> cassette (±gfp⁻¹ cargo) into a heat inducible derivative of λ^{LO} with sufficient cargo capacity to accommodate the 10.3 kb of the complete Pro-AG machinery (λ^{LO}Pro-AG vector—FIG. 21d). λ^{LO}Pro-AG is designed to launch the <gRNA-bla, −1 frame-shifted gfp> cassette into plasmid-borne bla target genes (FIG. 21c). A similar λ-phage was successfully programmed to target antibiotic resistance genes using standard CRISPR components²⁵. A key advantage of system of the invention is the expected increase in efficiency mediated by gRNA self-amplification. After establishing clonal λ^{LO}Pro-AG strains of E. coli, the lysogen is induced (e.g., by shifting to 42° C. for 15 minutes or by adding mitomycin-C) and dense cultures are harvested of the λPro-AG vector (>10⁹ per ml). Mid-log stage recipient cultures are then infected (1×10⁵ CFU) carrying AR plasmids (either high or intermediate copy number duel AR plasmids—FIG. 21c) with λ^{LO}Pro-AG phage at various multiplicities of infection (MOI—measured by plaque assay), induce (or not for controls) the Pro-AG system with aTC (Cas9) and arabinose (λRed) and measure total CFU to assess overall AR reduction as well as the proportion of CFU that remain Amp^{R} (targeted locus) versus Gm^{R} (control AR locus for p-ET) or Km^{R} (control for pSV3B05 plasmid) to assess editing efficiencies. The Pro-AG system may also be imported into phage ΦEB49 (ΦEB49Pro-AG), which has a similar genome size and organization as lambda and efficiently transduces UPEC. These experiments provide an estimate for the relative efficiencies of phage versus conjugal systems in disseminating Pro-AG components and in reducing AR. whether adding att phage integration sites into the MobV conjugal plasmid may also be tested, which combines the two HGT systems allowing the phage both to insert into the genome (lysogen) and to move with the MobV plasmid, results in synergistic reductions in AR. Since bacterial mutations conferring resistance to either phage or conjugal systems are likely to arise and limit delivery of Pro-AG components, developing these two complementary systems may reduce the impact of this anticipated difficulty.

### Methods

### Strains and Culture Conditions

Escherichia coli strain MG1655 WT and ΔRecA were provided by the B. Palsson and Susan Lovett Laboratories, respectively. Liquid cultures of E. coli were grown in Luria broth (LB) medium. When appropriate, antibiotics were added as following: chloramphenicol (Cm, 25 μg/ml), ampicillin (Amp, 100 μg/ml), spectinomycin (Spm, 50 μg/ml) and gentamicin (Gm, 10 μg/ml).

### Plasmid Construction

All constructs used in this study are listed in Table 1 with primer sequences provided in Table 2. Plasmids pKDsgRNA (pCRISPR) and pCas9-CR4 (pCas9) (Table 1) were purchased from Addgene (Cambridge, Mass.). pCRISPR(AmpgRNA1), pCRISPR (AmpgRNA2) and pCRISPRlacZ were built as previously described⁹. The 20 bp targeting sequences of the gRNAs were cloned into pKDsgRNA using circular polymerase extension cloning (CPEC)²³ of two linear PCR fragments (F1, 3kb; F2, 4kb). For pCRISPR(AmpgRNA1) (Table 1), F1 and F2 were obtained using the paired primers 13_FwAmpgRNA1/14_RvF1 and 15_FwF2/16_RvAmpgRNA1 (Table 2), respectively. For pCRISPR(AmpgRNA2) (Table 1), F1 and 1 and 2 were obtained using the paired primers 17_FwAmpgRNA2/14_RvF1 and 18_RvAmpgRNA2/15_FwF2 (Table 2), respectively. For pCRISPRlacZ (Table 1), F1 and F2 were obtained using the paired primers 33_FwlacZgRNA/14_RvF1 and 34_RvlacZgRNA/15_FwF2 (Table 2), respectively. PCR products with ˜280 bp of overlapping homology sequences and 20 bp of overlap in the protospacer region were DpnI digested for at least 15 min then gel-purified. Fragments 1 and 2 were mixed together in equal amounts (200 ng each) and CPEC cloned with 15 cycles and Phusion High-Fidelity Polymerase (NEB). 2.5 μl of the mixture was used to transform one-shot Stbl3 chemically competent E. coli cells (Thermo Fisher).

Plasmids expressing Pro-AG configurations pPro-AG(Amp), pPro-AG(lacZ) and pPro-AG Super-Cos (Table 1) were built in two Gibson (NEBuilder, NEB) assembly steps using two linear PCR fragments with flanking overlapping sequences. For the pPro-AG(Amp) plasmid construct, the first step consisted of cloning bla sequence homology arm 1 in pCRISPR(AmpgRNA2) to generate the plasmid pCRISPR(AmpgRNA2+HA1) (Table 1) by amplification of two PCR fragments (F1 and F2) with paired primers 37_FwgRNA2/38_RvgRNA2 and 39_FwHA1Amp/40_RvHA1Amp (Table 2), respectively. A second step consisted of cloning bla sequence homology arm 2 in pCRISPR(AmpgRNA2+HA1) to generate plasmid pPro-AG(Amp) (Table 1) by amplification of two PCR fragments (F1 and F2) with paired primers 41_FwgAmpHA1/42_RvgAmpHA1 and 43_FwHA2/44_RvHA2 (Table 2), respectively. For the pPro-AG(lacZ) plasmid construct, a first step consisted of cloning lacZ sequence homology arm 1 in pCRISPR(lacZ) to generate pCRISPR(lacZ+HA1) plasmid (Table 2) by amplification of two PCR fragments (F1 and F2) with paired primers 51_FwlacZgRNA/52_RvlacZgRNA and 53_FwHA1lacZ/54_RvHA1lacZ (Table 2), respectively. A second step consisted of cloning lacZ sequence homology arm 2 in pCRISPR(lacZ+HA1) to generate plasmid pPro-AG(lacZ) (Table 1) by amplification of two PCR fragments (F1 and F2) with paired primers 55 FwlacZHA1/56_FwlacZHA1 and 57_FwlacZHA2/58_RvlacZHA2 (Table 2), respectively. For the pPro-AG Super-Cos plasmid construct, bla sequence homology arm 1 was first cloned in pCRISPR(AmpgRNA2) to generate the plasmid pCRISPR(AmpgRNA2+SV3B05HA1HA1) (Table 1) by amplification of two PCR fragments (F1 and F2) with paired primers 106_FwHA1Supercos/107_RvHA1Supercos and 108_FwpKDsAmp2/109_RvpKDsAmp2 (Table 2), respectively. A second step consisted of cloning bla sequence homology arm 2 in pCRISPR(AmpgRNA2+SV3B05HA1HA1) to generate plasmid pPro-AG Super-Cos (Table 1) by amplification of two PCR fragments (F1 and F2) with paired primers 110_Fw HA2Supercos/111_Rev HA2Supercos and Pr_112Fw CosHA1/Pr_113Rv CosHA1 (Table 2), respectively.

Plasmids pETg, pPro-AGFP(Amp), pPro-AGFP(lacZ), pETgCRISPR and pETgPro-AG (Table 1) were generated by amplification of two linear PCR fragments (F1 and F2), with flanking overlapping sequences for Gibson assembly (NEBuilder, NEB). Paired primers 59_FwpET/60_RvpET and 61_FwGm/62_RvGm (Table 2) were used to amplify PCR F1 and F2 to assemble pETg. Paired primers 69_FwP-AG(Amp)/73_RvP-AG(Amp) and 72_FwGFP-Amp/68_RvGFP-Amp (Table 2) were used to amplify PCR F1 and F2 to assemble pPRO-AGFP(Amp). Paired primers 63_FwP-AG(lacZ)/64_RvP-AG(lacZ) and 65_FwGFP-lacZ/66_RvGFP-lacZ (Table 2) were used to amplify PCR F1 and F2 to assemble pPro-AGFP(lacZ). Paired primers 98_FwProAG/99-RvPro-AG and 100_FwpETg(Pro-AG)/101_RvpETg(Pro-AG) were used to amplify PCR F1 and F2 to assemble pETgCRISPR. Paired primers 102_FwCRISPR/103-RvCRISPR and 104_FwpETg(CRISPR)/105_RvpETg(CRISPR) were used to amplify PCR F1 and F2 to assemble pETgCRISPR.

For the pgRNAout (Amp) and pgRNAout (lacZ) plasmid constructs, pCRISPR(AmpgRNA2) and pCRISPR(lacZ), respectively were linearized with the NcoI restriction enzyme. Subsequently, three fragments with flanking overlapping sequences (F1, F2 and F3) were PCR amplified as follows. For pgRNAout (Amp) construct, homology arms 1 (F1) and 2 (F2) from pPro-AG(Amp) and gfp (F3) from plasmid #48138 (Addgene), using primer pairs 67_FwHA1-GFPout/68_RVHA1-GFPout, 69_FwHA2-GFPout/70_RvHA2-GFPout and 71_FwGFP-GFPout/72_RvGFP-GFPout (Table 2), respectively. For pgRNAout (lacZ) construct, homology arms 1 (F1) and 2 (F2) from pPro-AG(lacZ) and gfp (F3) from plasmid #48138 (Addgene), using primer pairs 88_FwHA1-lacZout/89_RvHA1-lacZout, 92_FwHA2-lacZout/93_RvHA2-lacZout and 90_FwGFP-lacZout/91_RvGFP-lacZout (Table 2), respectively. Gibson assembly was carried out with the linearized vectors and the three corresponding overlapping PCR fragments to generate pgRNAout(Amp) and pgRNAout(lacZ) (Table 1). All Gibson assembly reactions were transformed into NEB 5-alpha competent E. coli cells.

E. coli Transformation

Competent cells were prepared as previously described (Short Protocols in Molecular Biology, Chapter 1). For all plasmid transformations, 50 μl of aliquoted cells were gently thawed on ice, followed by addition of plasmid DNA prepared by QIAprep Spin Miniprep Kit (Qiagen), with 20 ng of each plasmid added to the transformation mix. E. coli cells were electroporated with the 1 mm Gene Pulser cuvette (Bio-Rad) at 1.6 kV and immediately resuspended in 250 μl super optimal broth with catabolite repression (SOC) media. Cells were allowed to recover for 2 h at 30° C. (for cells transformed with pKDsgRNA derived plasmids) and for 1 h at 37° C. for cells transformed with pETg plasmid. Serial dilutions of cells were plated on LB with the corresponding antibiotic and they were incubated at 30° C. (48 h) or 37° C. (24 h), respectively.

### Induction of Cas9 and Lambda-Red Enzymes

Single E. coli colonies obtained on plates after transformation were resuspended in 60 μl LB, which served as inoculum for 5 ml LB overnight cultures (˜15 h) grown at 30° C. with shaking (200 rpm). When appropriate, Cas9 expression was induced in cells carrying the pCas9 plasmid (Table 1) by adding 100 ng/ml anhydrotetracycline (aTc, Abcam) to the broth media. Similarly, when desired, k-Red expression was induced in cells carrying pKdsgRNA derivative plasmids (Table 1) by adding 50 mM arabinose (arab, Sigma) to the broth media during editing steps (FIGS. 4a-4b).

E. coli Colony Counts

Colony forming units (CFU) were determined similarly to the miniaturized plating method described previously with small modifications. Briefly, 25 μl of overnight culture, and dilution series of 10⁻¹ to 10⁻⁸, were spotted in triplicate on LB plates containing the appropriate antibiotic selection and incubated overnight at 30° C. No notable differences were observed when arab or aTc were added to the plates for k-Red or Cas9 induction, respectively.

### Sequence Analysis of Editing Events

For bla editing events, pETg plasmids and Super-Cos (SV3B06) cosmids were purified from Amp^{S}/Gm^{R} Amp^{S}/Km^{R}, respectively, single edited E. coli colonies by QIAprep Spin Miniprep Kit (Qiagen) and sequenced (Genewiz) with primer 35_FwAmp(ext) (Table 2). pET-derivative plasmids from single escapers were analyzed following the same parameters. For lacZ editing events, white E. coli colonies where quantified among the total white+blue mixed population of colonies on LB plates containing 1 mM IPTG and 0.03% (v/v) Bluo-Gal (Teknova). Single white colonies were grown overnight at 37° C. and total genomic DNA isolated by DNeasy Blood and Tissue (Qiagen). PCR products were obtained with Q5 DNA polymerase (NEB) and using primer pairs 49_FwlacZseq and 50_RvlacZseq, followed by sequencing analysis (Genewiz) with primer 63_RvlacZint (Table 2). Escaper blue colonies were also analyzed following the same parameters. gRNA plasmid constructs from escapers were sequenced with primers 27_Fw pKDSgRNAseq and 28_Rv pKDSgRNAseq.

### Visualization and Quantification of GFP Expression

10 μl aliquots from overnight cultures of E. coli exposed to the Pro-AGFP configuration in LB+1 mM IPTG and under Cas9 and λ-Red induction were mounted onto slides with coverslips. GFP fluorescence was visualized using a Zeiss Axio Observer.D1 fluorescence microscope. For GFP fluorescence quantification, single white colonies from E. coli lacZ editing plates were homogenized in 200 μl PBS and subsequently transferred to a 96 well plate. Optical density at 600ₙₘ and GFP fluorescence at 510ₙₘ were measured by using an EnSpire Plate Reader (PerkinElmer).

Since including GFP cargo sequences in the Pro-AG system for insertion into a high copy number plasmid (FIG. 3b) had no obvious impact on the copying efficiency relative to that observed with the minimal gRNA-only cassette (FIGS. 2b and 3b), it was speculated that this system also might be adapted for inserting cargo into single-copy sequences in the bacterial chromosome. Such enhancement of currently available precise genome editing tools¹⁸ could be useful for a wide variety of bacterial engineering applications. An analogous set of plasmids were constructed to those described above for evaluating CRISPR-control versus Pro-AG protocols in the context of editing the lacZ chromosomal target gene (FIG. 13a). Comparing efficiencies of copying a minimal “gRNA-lacZ-only” versus a larger “gRNA-lacZ:GFP” cassette into the lacZ target site (FIG. 13a, Table 1), a similar selection and induction procedure were followed, described above for the three plasmid strategy (FIGS. 4a-4b). In the context of the single-copy lacZ chromosomal target, induction of Cas9 following initial bacterial transformation resulted in a marked decrease in viable E. coli CFU following either the CRISPR-control or Pro-AG protocols (FIG. 13b). These profound reductions in recovered CFU are consistent with previously reported findings on Cas9-induced cleavage of single copy genomic targets that presumably reflect the lethal effects of unrepaired chromosomal double-stranded breaks¹⁹. Similarly, overnight cultures seeded from single colonies achieved significantly lower cell densities following induction of Cas9 alone or together with λRed than observed without Cas9 induction (FIG. 13c). Consistent with the lethal DNA-break hypothesis, induction of the λRed DNA repair cassette together with Cas9 significantly increased CFU recovery (FIG. 13d). Following induction of Cas9 alone, blue colonies comprised the overwhelmingly phenotype recovered on IPTG/X-Gal plates (intact lacZ gene function) (FIG. 13e), suggesting that Cas9 had not acted on the target sequences in these escaper colonies. Indeed, all tested CRISPR and Pro-AG induced blue colony escapers recovered following induction of Cas9 alone carried fully intact lacZ target sequences (FIG. 13a, bottom scheme). Induction of Cas9+λRed applied to the CRISPR-control regimen only rarely produced white colonies (˜2% of total colonies recovered, FIG. 14a). In such white colonies, lacZ target sequences were unable to be amplified by PCR, perhaps reflecting large Cas9-induced deletion events (FIG. 9b, top scheme), as observed in prior studies²⁰. In contrast, induction of Cas9+λRed in the Pro-AG and Pro-AGFP regimens led to recovery of ˜90% white colonies, suggesting lacZ inactivation by precise insertion of the homology flanked DNA cassettes (FIG. 9a). Indeed, all analyzed white colonies contained perfect gene-disrupting insertions of the gRNA-lacZ-only or gRNA-lacZ:GFP cargo cassettes into the gRNA-lacZ directed Cas9 cleavage site in the genomic lacZ locus (FIG. 9b, middle and bottom schemes, respectively), mirroring prior results with the high copy plasmid. Because the GFP coding sequences were designed to be in-frame with lacZ, these Pro-AGFP edited bacteria also expressed the fluorescent GFP marker upon simultaneous induction of the lac operon with IPTG and activation of Cas9+λRed (FIGS. 14c and 14d). Thus, the Pro-AG system is well suited for high efficiency precision editing and delivery of DNA cargo into a chromosomal locus. Sequencing of the gRNA constructs from Pro-AG escaper (blue) colonies following Cas9+λRed induction revealed deletions in the tet operator region and sequence insertion downstream HA1 in 100% of clones analyzed (FIG. 10b), suggesting escape dependent on inactivation of the gRNA donor plasmid.

Dosage amplification of gRNA2 accompanied its copying from its low-copy number launch plasmid to its high-copy plasmid targets, and experiments in which the gRNA component was either included in the amplifying cassette (gRNA-In) or left outside of the cassette (gRNA-Out) revealed that the enhanced efficiency of the Pro-AG system depended on the gRNA being placed within the homology arms cassette (FIG. 3a). In contrast, when targeting a single-copy E. coli chromosomal locus, it was hypothesized that the absence of gRNA amplification in this context would translate to equal efficiencies of the gRNA-in and gRNA-out configurations. The Pro-AGFP (gRNA-In) versus gRNA-out configurations were compared for insertion of the GFP cargo gene into E. coli chromosomal lacZ locus (FIG. 15a) and observed that both configurations performed similarly, with no significant differences in CFUs recovered following parallel editing protocols (FIG. 15b). Induction of Cas9+λRed applied to gRNA-Out configuration yielded more >90% edited colonies (FIG. 15c), with perfect insertion of GFP cargo into Cas9 cleavage site in the genomic lacZ locus at similar efficiency to the Pro-AGFP (gRNA-In) strategy (FIG. 15d).

TABLE 1
Plasmids used in this study

| Plasmid | Relevant genotype or phenotype^{α} | Source |
|---|---|---|
| pKDsgRNA | Spm^{r}, oripSC101, low copy number vector expressing the gRNA scaffold | Reference |
| | from the tet promoter and λ-red from the araBAD promoter | 9 |
| pCas9-CR4 or | Cm^{r}, orip15A, low copy number vector expressing cas9 from the tet | Reference |
| pCas9 | promoter | 9 |
| pET-Duet1 | Amp^{r}, oriF1, lacl⁺, lacZα, T7 promoter, lac operator, multi | (Novogen) |
| | copy cloning and expression vector. | |
| pETg | Amp^{r}, Gm^{r}, pET-Duet1 derivative, expressing Gm^{r} cassette from the pGm | This work |
| | promoter | |
| pCRISPR | Spm^{r}, pKDsgRNA derivative expressing AmpgRNA1 from the tet promoter; | This work |
| (AmpgRNA1) | carries 20 nucleotides (5’ TTACTTCTGACAACGATCGG3 ’) to direct Cas9 | |
| | cleavage of Amp^{r} cassette in pET and/or pET-g plasmid targets | |
| pCRISPR | Spm^{r}, pKDsgRNA derivative, expressing AmpgRNA2 from the tet promoter; | This work |
| (AmpgRNA2) | carries 20 nucleotides (5’ ATCGAACTGGATCTCAACAG 3’) to direct Cas9 | |
| | cleavage of Amp^{r} cassette in pET and/or pET-G | |
| pCRISPR | Spm^{r}, pCRISPR(AmpgRNA2) derivative; carries bla sequence homology arm | This work |
| (AmpgRNA2 + HA1) | 1 upstream AmpgRNA2 cassette | |
| pPro-AG(Amp) | Spm^{r}, pCRISPR(AmpgRNA2 + HA1) derivative; carries AmpgRNA2 cassette | This work |
| | flanked by bla sequence homology arms 1 and 2 | |
| pPro-AGFP(Amp) | Spm^{r}, pPRO-AG derivative; carries AmpgRNA2 cassette flanked by bla | This work |
| | sequence homology arms and gfp sequence flanked by homology arm 1 and | |
| | AmpgRNA2. | |
| pgRNAout (Amp) | Spm^{r}, pCRISPR(AmpgRNA2) derivative, carries gfp sequence flanked by | This work |
| | bla sequence homology arms (500 pb each) in a different location to | |
| | the one for AmpgRNA2 cassette | |
| pCRISPR(lacZ) | Spm^{r}, pKDsgRNA derivative expressing lacZgRNA from the tet promoter; | This work |
| | carries 20 nucleotides (5’ TGGAAGATCAGGATATGTGG3 ’) to direct Cas9 | |
| | cleavage of lacZ in the E. coli MG1655 chromosome. | |
| pCRISPR | Spm^{r}, pCRISPR(lacZ) derivative, carries lacZ sequence homology arm 1 | This work |
| (lacZ + HA1) | upstream lacZgRNA cassette | |
| pPro-AG(lacZ) | Spm^{r}, pCRISPR(lacZ + HA1) derivative; carries lacZgRNA cassette | This work |
| | flanked by lacZ sequence homology arms 1 and 2 | |
| pPro-AGFP | Spm^{r}, pPRO-AG derivative; carries lacZgRNA cassette flanked by lacZ | This work |
| (lacZ) | sequence homology arms and gfp sequence flanked by homology arm 1 and | |
| | lacZgRNA | |
| pETgCRISPR | Amp^{r}, Gm^{r}, pETg derivative; carries AmpgRNA2 cassette | This work |
| pETgPro-AG | Amp^{r}, Gm^{r}, pETg derivative; carries AmpgRNA2 cassette flanked by bla | This work |
| | sequence homology arms 1 and 2 | |
| pCRISPR | Spm^{r}, pCRISPR(AmpgRNA2) derivative; carries bla sequence homology arm | This work |
| (AmpgRNA2 + | 1 of SV3B05 upstream AmpgRNA2 cassette | |
| SV3B05HA1) | | |
| Super-Cos | Amp^{r}, Km^{r}, SuperCos derivative (Agilent); carries 40,647 bp from | N. Tschowri |
| (SV3B06) | Streptomyces venezuelae | Lab |
| pPro-AG | Spm^{r}, pCRISPR(AmpgRNA2 + SV3B05HA1) derivative; carries AmpgRNA2 | This work |
| Super-Cos | cassette flanked by bla sequence homology arms 1 and 2 of SV3B05 | |
| pgRNAout | Spm^{r}, pCRISPR(lacZ) derivative, carries gfp sequence flanked by lacZ | This work |
| (lacZ) | sequence homology arms (500 pb each) in a different location to the | |
| | one for lacZgRNA cassette | |

TABLE 2
OLIGONUCLEOTIDES USED IN THIS STUDY

| Primer ID | Sequence 5′-3′ | Use |
|---|---|---|
| 59_FwpET | aaactcccacatggattcgaaatcaatctaaagtatatatgagtaaacttggtctga | Amplification of 5458 bp (F1), spanning the pET vector, used for |
| | cag | Gibson assembly of pETG. |
| 60_RvpET | cccaagtaccgccacctaaaaaacttcatttttaatttaaaaggatctaggtgaaga | |
| | tcc | |
| 61_FwGm | taaattaaaaatgaagttttttaggtggcggtacttggg | Amplification of 656 bp (F2), spanning Gm cassette, used for |
| 62_RvGm | tatatactttagattgatttcgaatccatgtgggagtttattcttgac | Gibson assembly of pETG |
| 13_FwAmpgRNA1 | ttacttctgacaacgatcgggttttagagctagaaatagcaag | Amplification of 2868 bp (F1) to construct |
| 14_RvF1 | tttataacctccttagagctcga | pCRIS PR(AmpgRNA 1) |
| 15_FwF2 | ccaattgtccatattgcatca | Amplification of 4434 bp (F2) to construct |
| 16_RvAmpgRNA1 | ccgatcgttgtcagaagtaagtgctcagtatctctatcactga | pCRIS PR(AmpgRNA 1) |
| 17_FwAmpgRNA2 | atcgaactggatctcaacaggttttagagctagaaatagcaag | Paired with primer 14 for amplification of 2868 bp (F1) to |
| | | construct pCRISPR(AmpgRNA2) |
| 18_RvAmpgRNA2 | ctgttgagatccagttcgatgtgctcagtatctctatcactga | Paired with primer 15 for amplification of 4434 bp (F2) to |
| | | construct pCRISPR(AmpgRNA2) |
| 37_FwgRNA2 | tacatcgaactggatctcaacttccctatcagtgatagagattgacat | Amplification of 7002 bp (F1), spanning pCRISPR(AmpgRNA2) |
| 38_RvgRNA2 | agccgtagttaggccaccacaattccagaaatcatccttagcgaaagc | for Gibson assembly of pCRISPR(AmpgRNA2 + HA1) |
| 39_FwHA1Amp | taaggatgatttctggaattgtggtggcctaactacggc | Amplification of bla sequence HAI (540 bp, F2) for Gibson |
| 40_RvHA1Amp | ctctatcactgatagggaagttgagatccagttcgatgtaacccact | assembly of pCRISPRgRNA2 + HA1 |
| 41_FwgAmpHA1 | ggcggataaagttgcaggacttgggcccgaacaaaaact | Amplification of 7502 bp (F1), spanning pCRISPR(gRNA2 + HA1) |
| 42_RvgAmpHA1 | tctcaaggatcttaccgctggcttcaaaaaaagcaccgactcg | for Gibson assembly of pPro-AG(Amp) |
| 43_FwHA2 | gtcggtgctttttttgaagccagcggtaagatccttgagagttt | Amplification of bla sequence HA2 (540 bp, F2) for Gibson |
| 44_RvHA2 | gagtttttgttcgggcccaagtcctgcaactttatccgcct | assembly of pPro-AG(Amp) |
| 69_FwP-AG(Amp) | tggacgagctgtacaagtaacttccctatcagtgatagagattgaca | Amplification of 8001 bp (F1), spanning pCRISPR(gRNA2 + HA1) |
| 73_RvP-AG(Amp) | cctcgcccttgctcaccatttgagatccagttcgatgtaacccact | for Gibson assembly of pPro-AGFP(Amp) |
| 72_FwGFP-Amp | tacatcgaactggatctcaaatggtgagcaagggcgag | Amplification of gfp sequence (760 bp, F2) for Gibson assembly |
| 68_RvGFP-Amp | ctctatcactgatagggaagttacttgtacagctcgtccatgccg | of pPro-AGFP(Amp) |
| 33_FwLacZgRNA | tggaagatcaggatatgtgggttttagagctagaaatagcaag | Paired with primer 14 for amplification of 2868 bp (F1) to |
| | | construct pCRISPR(lacZ) |
| 34_RvLacZgRNA | ccacatatcctgatcttccagtgctcagtatctctatcactga | Paired with primer 15 for amplification of 4434 bp (F2) to |
| | | construct pCRISPR(lacZ) |
| 51_FwlacZgRNA | atctggaagatcaggatatgcttccctatcagtgatagagattgacatcc | Amplification of 7002 bp (F1), spanning pCRISPR(lacZgRNA) |
| 52_RvlacZgRNA | ggcctcttcgctattacgccaattccagaaatcatccttagcgaaagc | for Gibson assembly of pCRISPR(lacZgRNA + HA1) |
| 53_FwHA1lacZ | taaggatgatttctggaattggcgtaatagcgaagaggcc | Amplification of lacZ sequence HAI (540 bp, F2) for Gibson |
| 54_RvHA1lacZ | ctctatcactgatagggaagcatatcctgatcttccagataactgccg | assembly of pCRISPR(lacZgRNA + HA1) |
| 55_FwlacZHA1 | atggtcaggtcatggatgagttgggcccgaacaaaaactcatctcag | Amplification of 7502 bp spanning pCRISPR(lacZgRNA + HA1) |
| 56_FwlacZHA1 | aaaatgccgctcatccgccagcttcaaaaaaagcaccgact | for Gibson assembly of pPro-AG(lacZ) |
| 57_FwlacZHA2 | gtcggtgctttttttgaagctggcggatgagcggca | Amplification of lacZ sequence HA2 (541 bp, F2) for Gibson |
| 58_RvlacZHA2 | gagtttttgttcgggcccaactcatccatgacctgaccatgc | assembly of pPro-AG(lacZ) |
| 63_FwP-AG(lacZ) | tggacgagctgtacaagtaacttccctatcagtgatagagattgacatcc | Amplification of 8003 bp (F1), spanning pCRISPR(lacZgRNA + HA1) |
| 64_RvP-AG(lacZ) | tcctcgcccttgctcaccatcatatcctgatcttccagataactgccg | for Gibson assembly of pPro-AGFP(lacZ) |
| 65_FwGFP-lacZ | atctggaagatcaggatatgatggtgagcaagggcgag | Amplification of gfp sequence (760 bp, F2) for Gibson assembly |
| 66_RvGFP-lacZ | ctctatcactgatagggaagttacttgtacagctcgtccatgccg | of pPro-AGFP(lacZ) |
| 67_FwHA1-GFPout | accagtagaaacagacgaagaatcgtggtggcctaactacggct | Amplification of bla sequence HAI (535 bp, F1) for Gibson |
| 68_RvHA1-GFPout | ttgctcaccatttgagatccagttcgatgtaacccac | assembly of pgRNAout(Amp) |
| 69_FwHA2-GFPout | ctgtacaagtaacagcggtaagatccttgagagtttt | Amplification of bla sequence HA2 (537 bp, F2) for Gibson |
| 70_RvHA2-GFPout | tatcaaagggaaaactgtccataccgtcctgcaactttatccgcct | assembly of pgRNAout(Amp) |
| 7l_FwGFP-GFPout | ggatctcaaatggtgagcaagggcgag | Amplification of gfp sequence (742 bp, F3) for Gibson assembly |
| 72_RvGFP-GFPout | atcttaccgctgttacttgtacagctcgtccatgccg | of pgRNAout(Amp) |
| 49_FwlacZseq | agacgcgaattatttttgatggcgttaactcg | Amplification of lacZ internal sequence for sequencing editing |
| 50_RvlacZseq | gaagggctggtcttcatccacgcg | events. PCR products length varied among insertions (2008 bp for |
| | | GFP cargo delivered, 1288 bp for gRNA cassette insertion and |
| | | 1124 bp for escapers) |
| 63_RvlacZint | gccgctggcgacctgcgtttca | Sequencing of lacZ editing events, internal primer that hybridizes |
| | | within lacZ HA2 and downstream lacZ gRNA sequences |
| 35_FwAmp(ext) | gagtattcaacatttccgtgtcgc | Sequencing of bla editing events, internal primer that hybridizes |
| | | within bla HAI and downstream AmpgRNA2 sequences |
| 102_FwCRISPR | cagggtagccagcagcatcccttccctatcagtgatagagattgacatcc | Amplification of AmpgRNA2 cassette (203 bp, F1) for Gibson |
| 103_RvCRISPR | tgttccggatctgcatcgcagcttcaaaaaaagcaccgactcg | assembly of pETgCRISPR |
| 104_FwpETg(CRISPR) | gtcggtgctttttttgaagctgcgatgcagatccggaac | Amplification of 6076 bp (F2), spanning pETg for Gibson |
| 105_RvpETg(CRISPR) | ctctatcactgatagggaagggatgctgctggctaccc | assembly of pETgCRISPR |
| 98_FwPro-AG | cagggtagccagcagcatccgtggtggcctaactacggct | Amplification of Pro-AG cassette (1204 bp, F1) for Gibson |
| 99_RvPro-AG | tgttccggatctgcatcgcagtcctgcaactttatccgcct | assembly of pETgPro_AG |
| 100_FwpETg(Pro-AG) | ggcggataaagttgcaggactgcgatgcagatccgga | Amplification of 6076 bp (F2), spanning pETg for Gibson |
| 101_RvpETg(Pro-AG) | agccgtagttaggccaccacggatgctgctggctaccct | assembly of pETgCRISPR |
| 106_FwHA1Supercos | ctctatcactgatagggaagttgagatccagttcgatgtaaccc | Amplification of bla sequence HAI from Super-Cos (SV3B05) |
| 107_RvHA1Supercos | taaggatgatttctggaatttgaggacgaggtggcc | (540 bp, F1) for Gibson assembly of pCRISPR |
| | | (AmpgRNA2 + SV3B05HA1) |
| 108_FwpKDsAmp2 | tacatcgaactggatctcaacttccctatcagtgatagagattgacat | Amplification of 7002 bp (F2), spanning pCRISPR(AmpgRNA2) |
| 109_RvpKDsAmp2 | tgacggccacctcgtcctcaaattccagaaatcatccttagcgaaag | for Gibson assembly of pCRISPR (AmpgRNA2 + SV3B05HA1) |
| 110_Fw HA2Supercos | gagtttttgttcgggcccaagtcctgcaactttatccgcct | Amplification of bla sequence HA2 from Super-Cos (SV3B05) |
| 111_Rev HA2Supercos | gtcggtgctttttttgaagccagcggtaagatccttgagagtttt | (540 bp, F1) for Gibson assembly of pPro-AG Super-Cos |
| Pr_112Fw CosHA1 | ggcggataaagttgcaggacttgggcccgaacaaaaact | Amplification of 7502 bp (F2), spanning pCRISPR |
| Pr_113Rv CosHA1 | tctcaaggatcttaccgctggcttcaaaaaaagcaccgactcg | (AmpgRNA2 + SV3B05HA1) for Gibson assembly of pPro-AG |
| | | Super-Cos |
| 88_FwHA1-lacZout | tcaggtttgtgccaataccagtagaaacagacgaagaatcggcgtaatagcga | Amplification of lacZ sequence HAI (560 bp, F1) for Gibson |
| 89_RvHA1-lacZout | tcctcgcccttgctcaccatcatatcctgatcttccagataactgccg | assembly of pgRNAout(lacZ) |
| 90_FwGFP-lacZout | atctggaagatcaggatatgatggtgagcaagggcgag | Amplification of gfp sequence (760 bp, F2) for Gibson assembly |
| 91_RvGFP-lacZout | aaaatgccgctcatccgccattacttgtacagctcgtccatgcc | of pgRNAout(lacZ) |
| 92_FwHA2-lacZout | tggacgagctgtacaagtaatggcggatgagcggca | Amplification of lacZ sequence HA2 (559 bp, F3) for Gibson |
| 93_RvHA2-lacZout | gttcaccgttacatatcaaagggaaaactgtccataccctcatccatgacctgacc | assembly of pgRNAout(lacZ) |
| 27_Fw pKDSgRNAseq | atcccgtgacaggtcattcagactg | Sequencing of gRNA plasmid contract from editing events. |
| 28_Rv pKDSgRNAseq | gatttaatctgtatcagg | |

### REFERENCES

1 DiCarlo, J. E., Chavez, A., Dietz, S. L., Esvelt, K. M. & Church, G. M. Safeguarding CRISPR-Cas9 gene drives in yeast. Nat Biotechnol 33, 1250-1255, doi:10.1038/nbt.3412 (2015).
2 Gantz, V. M. & Bier, E. The mutagenic chain reaction: a method for converting heterozygous to homozygous mutations. Science 348, 442-444, doi:science.aaa5945 [pii] 10.1126/science.aaa5945 (2015).
3 Gantz, V. M. et al. Highly efficient Cas9-mediated gene drive for population modification of the malaria vector mosquito Anopheles stephensi. Proc Natl Acad Sci USA 112, E6736-6743, doi:1521077112 [pii] 10.1073/pnas.1521077112 (2015).
4 Hammond, A. et al. A CRISPR-Cas9 gene drive system targeting female reproduction in the malaria mosquito vector Anopheles gambiae. Nat Biotechnol 34, 78-83, doi:10.1038/nbt.3439 (2016).
5 Grunwald, H. A. et al. Super-Mendelian inheritance mediated by CRISPR-Cas9 in the female mouse germline. Nature 566, 105-109, doi:10.1038/s41586-019-0875-2 (2019).
6 Gratz, S. J. et al. Highly specific and efficient CRISPR/Cas9-catalyzed homology-directed repair in Drosophila. Genetics 196, 961-971, doi:10.1534/genetics.113.160713 (2014).
7 Javed, M. R. et al. CRISPR-Cas System: History and Prospects as a Genome Editing Tool in Microorganisms. Curr Microbiol 75, 1675-1683, doi:10.1007/s00284-018-1547-4 (2018).
8 Jiang, W., Bikard, D., Cox, D., Zhang, F. & Marraffini, L. A. RNA-guided editing of bacterial genomes using CRISPR-Cas systems. Nat Biotechnol 31, 233-239, doi:10.1038/nbt.2508 (2013).
9 Reisch, C. R. & Prather, K. L. J. Scarless Cas9-assisted recombineering (no-SCAR) in Escherichia coli, an easy-to-use system for genome editing. Curr Protoc Mol Biol 117, 31 38 31-31 38 20, doi:10.1002/cpmb.29 (2017).
10 Citorik, R. J., Mimee, M. & Lu, T. K. Sequence-specific antimicrobials using efficiently delivered RNA-guided nucleases. Nat Biotechnol 32, 1141-1145, doi:10.1038/nbt.3011 (2014).
11 Johnson, T. J. & Nolan, L. K. Pathogenomics of the virulence plasmids of Escherichia coli. Microbiol Mol Biol Rev 73, 750-774, doi:10.1128/MMBR.00015-09 (2009).
12 Partridge, S. R., Kwong, S. M., Firth, N. & Jensen, S. O. Mobile genetic elements associated with antimicrobial resistance. Clin Microbiol Rev 31, doi:10.1128/CMR.00088-17 (2018).
13 Bassalo, M. C. et al. Rapid and efficient one-step metabolic pathway integration in E. coli. ACS Synth Biol 5, 561-568, doi:10.1021/acssynbio.5b00187 (2016).
14 Cui, L. & Bikard, D. Consequences of Cas9 cleavage in the chromosome of Escherichia coli. Nucleic Acids Res 44, 4243-4251, doi:10.1093/nar/gkw223 (2016).
15 Murphy, K. C. lambda recombination and recombineering. EcoSal Plus 7, doi:10.1128/ecosalplus.ESP-0011-2015 (2016).
16 Thomason, L. C., Sawitzke, J. A., Li, X., Costantino, N. & Court, D. L. Recombineering: genetic engineering in bacteria using homologous recombination. Curr Protoc Mol Biol 106, 1 16 11-39, doi:10.1002/0471142727.mb0116s106 (2014).
17 Gantz, V. M. & Bier, E. The dawn of active genetics. Bioessays 38, 50-63, doi:10.1002/bies.201500102 (2016).
18 Kyrou, K. et al. A CRISPR-Cas9 gene drive targeting doublesex causes complete population suppression in caged Anopheles gambiae mosquitoes. Nat Biotechnol 36, 1062-1066, doi:10.1038/nbt.4245 (2018).
19 Ye, M. et al. A review of bacteriophage therapy for pathogenic bacteria inactivation in the soil environment. Environ Int 129, 488-496, doi:10.1016/j.envint.2019.05.062 (2019).
20 Pazda, M., Kumirska, J., Stepnowski, P. & Mulkiewicz, E. Antibiotic resistance genes identified in wastewater treatment plant systems—A review. Sci Total Environ 697, 134023, doi:10.1016/j.scitotenv.2019.134023 (2019).
21 El Haddad, L., Harb, C. P., Gebara, M. A., Stibich, M. A. & Chemaly, R. F. A systematic and critical review of bacteriophage therapy against multidrug-resistant ESKAPE organisms in hjumans. Clin Infect Dis 69, 167-178, doi:10.1093/cid/ciy947 (2019).
22 Hamilton, T. A. et al. Efficient inter-species conjugative transfer of a CRISPR nuclease for targeted bacterial killing. Nature Communications 10, 4544, doi:10.1038/s41467-019-12448-3 (2019).
23 Quan, J. & Tian, J. Circular polymerase extension cloning. Methods Mol Biol 1116, 103-117, doi:10.1007/978-1-62703-764-8_8 (2014).
24 Bikard, D., C. W. Euler, W. Jiang, P. M. Nussenzweig, G. W. Goldberg, X. Duportet, V. A. Fischetti, and L. A. Marraffini. Exploiting CRISPR-Cas nucleases to produce sequence-specific antimicrobials. 2014. Nat Biotechnol 32: 1146-50.
25 Yosef, I., M. Manor, R. Kiro, and U. Qimron. Temperate and lytic bacteriophages programmed to sensitize and kill antibiotic-resistant bacteria. 2015. Proc Natl Acad Sci USA 112: 7267-72.
26 Kim, J. S., D. H. Cho, M. Park, W. J. Chung, D. Shin, K. S. Ko, and D. H. Kweon. CRISPR/Cas9-Mediated Re-Sensitization of Antibiotic-Resistant Escherichia coli Harboring Extended-Spectrum beta-Lactamases. 2016. J Microbiol Biotechnol 26: 394-401.
27 Park, J. Y., B. Y. Moon, J. W. Park, J. A. Thornton, Y. H. Park, and K. S. Seo. Genetic engineering of a temperate phage-based delivery system for CRISPR/Cas9 antimicrobials against Staphylococcus aureus. 2017. Sci Rep 7: 44929.
28 Ram, G., H. F. Ross, R. P. Novick, I. Rodriguez-Pagan, and D. Jiang. Conversion of staphylococcal pathogenicity islands to CRISPR-carrying antibacterial agents that cure infections in mice. 2018. Nat Biotechnol 36: 971-976.
29 Rodrigues, M., S. W. McBride, K. Hullahalli, K. L. Palmer, and B. A. Duerkop. Conjugative Delivery of CRISPR-Cas9 for the Selective Depletion of Antibiotic-Resistant Enterococci. 2019. Antimicrob Agents Chemother 63.
30 Adolfi, A., V. M. Gantz, N. Jasinskiene, H.-F. Lee, K. Hwang, E. A. Bulger, A. Ramaiah, J. B. B. Bennett, G. Terradas, J. J. Emerson, J. M. Marshall, E. Bier, and A. A. James. Efficient population modification gene-drive rescue system in the malaria mosquito Anopheles stephensi. 2020. Nat Commun: 5553.
31 Carballar-Lejarazu, R., C. Ogaugwu, T. Tushar, A. Kelsey, T. B. Pham, J. Murphy, H. Schmidt, Y. Lee, G. C. Lanzaro, and A. A. James. Next-generation gene drive for population modification of the malaria vector mosquito, Anopheles gambiae. 2020. Proc Natl Acad Sci USA.
32 Simoni, A., A. M. Hammond, A. K. Beaghton, R. Galizi, C. Taxiarchi, K. Kyrou, D. Meacci, M. Gribble, G. Morselli, A. Burt, T. Nolan, and A. Crisanti. A male-biased sex-distorter gene drive for the human malaria vector Anopheles gambiae. 2020. Nat Biotechnol.
33 Valderrama, J. A., S. S. Kulkarni, V. Nizet, and E. Bier. A bacterial gene-drive system efficiently edits and inactivates a high copy number antibiotic resistance locus. 2019. Nature Communications 10: 5726.
34 Guichard, A., T. Haque, M. Bobik, X. S. Xu, C. Klanseck, R. B. S. Kushwah, M. Berni, B. Kaduskar, V. M. Gantz, and E. Bier. Efficient allelic-drive in Drosophila. 2019. Nat Commun 10: 1640.
35 Cooper, R. M., L. Tsimring, and J. Hasty. Inter-species population dynamics enhance microbial horizontal gene transfer and spread of antibiotic resistance. 2017. Elife 6.
36 Perez-Mendoza, D. and F. de la Cruz. Escherichia coli genes affecting recipient ability in plasmid conjugation: are there any? 2009. BMC Genomics 10: 71.

What is claimed is:

1. A method of inhibiting antibiotic resistance in bacteria comprising modifying a bacterial plasmid gene for antibiotic resistance with a prokaryotic-active genetics (Pro-AG) system comprising:
(a) a first plasmid encoding an inducible Cas9 protein; and
(b) a second plasmid encoding:
(i) a guide ribonucleic acid (gRNA) cassette comprising a promoter for constitutive expression of a gRNA having a sequence that hybridizes to a target genomic sequence on a target plasmid in the bacteria, wherein the target genomic sequence in the bacteria confers antibiotic resistance;
(ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and
(iii) an inducible λRed DNA repair cassette,
wherein inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby modifying the bacterial plasmid gene to inhibit antibiotic resistance.

2. The method of claim 1, wherein copies of the guide RNA cassette increase via a positive feedback loop allowing for self-amplification of the Pro-AG system.

3. The method of claim 1, wherein the first plasmid is present in a lower copy number than the second plasmid and the bacterial plasmid.

4. The method of claim 4, wherein the bacterial gene for antibiotic resistance is a beta-lactamase gene.

5. The method of claim 1, wherein the antibiotic is ampicillin or gentamicin.

6. The method of claim 1, wherein the bacteria is Escherichia coli.

7. The method of claim 1, wherein the Cas9 protein is induced with anhydrotetracycline.

8. The method of claim 1, wherein the λRed DNA repair cassette is induced with arabinose.

9. The method of claim 1, wherein a Tet promoter drives constitutive expression of the gRNA.

10. The method of claim 1, wherein the bacteria is in a subject.

11. The method of claim 1, wherein the bacteria is on a solid surface or in a liquid.

12. The method of claim 1, wherein the second plasmid further comprises at least one cargo sequence, which is inserted into the bacterial plasmid.

13. The method of claim 12, wherein the at least one cargo sequence encodes GFP.

14. The composition of claim 12, wherein the at least one cargo sequence is not flanked by the first and second homology sequences on the second plasmid.

15. The method of claim 1, wherein the second plasmid comprises a dual Pro-AG system further comprising,
(i) a further guide ribonucleic acid (gRNA) cassette comprising a further promoter for constitutive expression of a further gRNA having a further sequence that hybridizes to a further target genomic sequence on the target plasmid in the bacteria; and
(ii) a further first homology arm and a further second homology arm each flanking opposite ends of the further gRNA cassette in the second plasmid, wherein the further first homology arm and the further second homology arm have sequences that hybridize to respective sequences on opposite sides of a cut site for the Cas9 protein on the further target genomic sequence.

16. The method of claim 1, wherein the second plasmid comprises a nested Pro-AG system further comprising,
(i) a further gRNA having a further sequence that hybridizes to a further target genomic sequence on the target plasmid in the bacteria, wherein the further guide RNA is adjacent to the first homology arm outside the gRNA cassette; and
(ii) a further first homology arm outside the gRNA cassette on a side of the plasmid adjacent the second homology arm, and a further second homology arm outside the gRNA cassette on an opposite side of the plasmid adjacent the further gRNA, wherein the further first homology arm and the further second homology arm have sequences that hybridize to respective sequences on opposite sides of a cut site for the Cas9 protein on the further target genomic sequence.

17. The method of claim 1, wherein the second plasmid comprises an allelic Pro-AG system further comprising,
(i) a further gRNA having a further sequence that hybridizes to a further target genomic sequence on the target plasmid in the bacteria, wherein the further guide RNA is adjacent to the gRNA outside the gRNA cassette; and
(ii) a further first homology arm adjacent to and a further second homology arm outside the gRNA cassette, wherein the further first homology arm and the further second homology arm have sequences that hybridize to respective sequences on opposite sides of a cut site for the Cas9 protein on the further target genomic sequence.

18. A method of inhibiting a bacterial plasmid gene with a prokaryotic-active genetics (Pro-AG) system comprising:
(a) a first plasmid encoding an inducible Cas9 protein; and
(b) a second plasmid encoding:
(i) a guide ribonucleic acid (gRNA) cassette comprising a promoter for constitutive expression of a gRNA having a sequence that hybridizes to a target genomic sequence on a target plasmid in the bacteria;
(ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and
(iii) an inducible λRed DNA repair cassette,
wherein inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

19. The method of claim 18, wherein the gene confers antibiotic resistance.

20. The method of claim 18, wherein the gene encodes a virulence factor, a membrane transporters or an efflux pump.

21. A bacterial gene editing composition comprising a prokaryotic-active genetics (Pro-AG) system comprising:
(a) a first plasmid encoding an inducible Cas9 protein; and
(b) a second plasmid encoding:
(i) a guide ribonucleic acid (gRNA) cassette comprising a promoter for constitutive expression of a gRNA having a sequence that hybridizes to a target genomic sequence on a target plasmid in the bacteria;
(ii) a first homology arm and a second homology arm each flanking opposite ends of the gRNA cassette in the second plasmid, wherein the first homology arm and the second homology arm have sequences that hybridize to respective sequences on opposite sides of a cut site for the Cas9 protein on the target genomic sequence; and
(iii) an inducible λRed DNA repair cassette,
wherein inducing expression of the Cas9 protein effects association of the Cas9 with the gRNA to form an endonuclease complex and cleavage of the target genomic sequence at the cut site, and inducing expression of the λRed DNA repair cassette effects integration of a copy of the gRNA cassette into the cut site by homology directed repair, thereby inhibiting a bacterial plasmid gene.

22. The composition of claim 21, wherein the first plasmid further comprises a second guide RNA sequence, wherein the second guide RNA sequence targets a second target genomic sequence.

23. The composition of claim 22, wherein the second target genomic sequence is on the first plasmid, wherein the second guide RNA sequence is flanked by a third homology arm and a fourth homology arm on the first plasmid, and wherein the third homology arm and the fourth homology arm flank the second target genomic sequence on the target plasmid.

24. The composition of claim 22, further comprising a Pro-AG dependent genetic relay and switch circuit comprising:
a second target plasmid, wherein the second target genomic sequence is on the second target plasmid, wherein the first target plasmid comprises a third homology arm and a fourth homology arm, and when induced the third homology arm and the fourth homology arm flank the second target genomic sequence on the second target plasmid.

25. The composition of claim 24, wherein the first and second guide RNA sequences are first inserted into the first target plasmid and are then inserted into the second target plasmid.

26. The composition of claim 24, further comprising a Pro-AG amplifier system comprising an operator/promoter cargo in the gRNA cassette.
